(19) United States
(12) Patent Application Publication
(10) Pub. No.: US 2021/0214779 A1
(43) Pub. Date: Jul. 15, 2021

(54) ASSESSING HOST RNA USING ISOTHERMAL AMPLIFICATION AND RELATIVE ABUNDANCE

(71) Applicant: Inflammatix, Inc., Burlingame, CA (US)

(72) Inventors: David Rawling, San Mateo, CA (US); Timothy E. Sweeney, Redwood City, CA (US); Mark Eshoo, San Diego, CA (US)

(21) Appl. No.: 17/274,140

(22) PCT Filed: Sep. 18, 2019
(86) PCT No.: PCT/US2019/051765

§ 371 Date: Mar. 5, 2021

Related U.S. Application Data
(60) Provisional application No. 62/733,517, filed on Sep. 19, 2018.

(51) Int. Cl. C12Q 1/6851; G01N 21/64

(52) U.S. Cl. CPC C12Q 1/6851; G01N 2021/6441; G01N 21/6428

(57) ABSTRACT

A method for estimating a diagnostic score includes adding a first aliquot of a test sample to a first reaction vessel, and a second aliquot of the test sample to a second reaction vessel. The test sample includes a first target nucleic acid and a second target nucleic acid. The second target nucleic acid has a lower expected abundance than the first target nucleic acid. The second aliquot has a larger volume than the first aliquot. A first relative abundance value of the first target nucleic acid and a second relative abundance value of the second target nucleic acid are estimated by performing a first real-time quantitative isothermal amplification assay in the first reaction vessel, and performing a second real-time quantitative isothermal amplification assay in the second reaction vessel, respectively. The diagnostic score is estimated based on the first relative abundance value and the second relative abundance value.

### CROSS-REFERENCES TO RELATED APPLICATIONS

This application claims the benefit of U.S. Provisional Patent Application No. 62/733,517, filed on Sep. 19, 2018, the content of which is incorporated by reference in its entirety.

### FIELD OF THE INVENTION

The disclosure relates to methods of estimating a diagnostic score. More specifically, the disclosure relates to methods of estimating a diagnostic score using real-time quantitative isothermal amplification. A test sample includes a first target nucleic acid and a second target nucleic acid. A first relative abundance value of the first target nucleic acid and a second relative abundance value of the second target nucleic acid are estimated by performing real-time quantitative isothermal amplification assays. The diagnostic score is estimated based on the first relative abundance value and the second relative abundance value.

### BACKGROUND OF THE INVENTION

Quantitation of a target gene present in low abundance (e.g., low copy number) amongst other genes and nucleic acids in a sample can be difficult. Current methods of detection often involve amplification (e.g., polymerase chain reaction (PCR)) of the gene or a product related to transcription of the gene (e.g., mRNA). With the advent of reverse transcriptase (RT)-PCR, RNA molecules can be specifically targeted to forgo cDNA molecules. The resulting cDNA molecules can themselves be used as a template for amplification, readily improving detection of target genes or gene products in samples. Isothermal amplification methods offer faster amplification rates and lower complexity instruments. However, isothermal amplification techniques, such as real-time quantitative loop-mediated isothermal amplification (real-time qLAMP) based methods, may not reliably quantitate target nucleic acids below 1,000 copies of the target nucleic acid for mammalian RNA targets (see, Nixon et al., (2014), Bimolecular Detection and Quantitation, 2: 4-10).

Improved diagnostics for acute infections (e.g., bacterial and viral) could decrease morbidity and mortality rates. Yet, methods for detecting acute infections are lacking in specificity, sensitivity and speed. While some amplification techniques can detect pathogens directly from blood cultures, these tests tend to rely on select subsets of specific pathogens, meaning that some pathogens are not detected. Furthermore, many infections do not enter the bloodstream, and so are not detectable by non-invasive methods. As such, there remains a need to evaluate host responses (e.g., host gene expression) to classify bacterial and viral infections, and correlate the host response to outcomes such as diagnosis and/or treatment regimens.

### BRIEF SUMMARY OF THE INVENTION

Provided herein is a method for estimating a diagnostic score of a test sample using real-time quantitative isothermal amplification. In some embodiments, the method includes obtaining the test sample containing at least a first target nucleic acid and at least a second target nucleic acid, and a reference nucleic acid. Each of the first target nucleic acid, the second target nucleic acid, and the reference nucleic acid comprises a mammalian host nucleic acid. The method further includes adding a first aliquot of the test sample to a first reaction vessel for quantitative isothermal amplification of the first target nucleic acid, and adding a second aliquot of the test sample to a second reaction vessel for quantitative isothermal amplification of the second target nucleic acid. Each of the first reaction vessel and the second reaction vessel contains a master mix for isothermal amplification of the first target nucleic acid, the second target nucleic acid, and the reference nucleic acid. The second target nucleic acid has a lower expected abundance than the first target nucleic acid in the test sample. The first aliquot has a first volume. The second aliquot has a second volume greater than the first volume. The method further includes performing a first real-time quantitative isothermal amplification assay in the first reaction vessel by: starting a first reaction in the first reaction vessel; determining a first time-to-threshold value for the first target nucleic acid in the first reaction; determining a first reference time-to-threshold value for the reference nucleic acid in the first reaction; and estimating a first relative abundance value of the first target nucleic acid in the test sample relative to the reference nucleic acid based at least on the first time-to-threshold value and the first reference time-to-threshold value. The method further includes performing a second real-time quantitative isothermal amplification assay in the second reaction vessel by: starting a second reaction in the second reaction vessel; determining a second time-to-threshold value for the second target nucleic acid in the second reaction; determining a second reference time-to-threshold value for the reference nucleic acid in the second reaction; and estimating a second relative abundance value of the second target nucleic acid in the test sample relative to the reference nucleic acid based at least on the second time-to-threshold value and the second reference time-to-threshold value. The method further includes estimating the diagnostic score of the test sample based on the first relative abundance value for the first target nucleic acid and the second relative abundance value for the second target nucleic acid.

In another aspect, provided herein is a method of estimating a diagnostic score using real-time quantitative isothermal amplification. In some embodiments, the method includes obtaining a test sample from a mammalian subject. The test sample contains at least a first target nucleic acid and at least a second target nucleic acid, and a reference nucleic acid. Each of the first target nucleic acid, the second target nucleic acid, and the reference nucleic acid has an expected concentration in the test sample that is within a dynamic range of the real-time quantitative isothermal amplification as verified over a cohort population of interest. The method further includes adding an aliquot of the test sample to at least one reaction vessel containing a master mix for isothermal amplification of the first target nucleic acid, the second target nucleic acid, and the reference nucleic acid; starting at least one reaction of isothermal amplification in the at least one reaction vessel; determining a first time-to-threshold value for the first target nucleic acid in the at least one reaction; determining a second time-to-threshold value for the second target nucleic acid in the at least one reaction; determining a reference time-to-threshold value for the reference nucleic acid in the at least one reaction; estimating a first relative abundance value of the first target nucleic acid relative to the reference nucleic acid in the test sample based at least on the first time-to-threshold value and the reference time-to-threshold value; estimating a second relative abundance value of the second target nucleic acid relative to the reference nucleic acid in the test sample based at least on the second time-to-threshold value and the reference time-to-threshold value; and estimating the diagnostic score of the test sample based on the first time-to-threshold value for the first target nucleic acid and the second time-to-threshold value for the second target nucleic acid.

In another aspect, provided herein is a method for estimating a diagnostic score by performing real-time quantitative isothermal amplification on a test sample. In some embodiments, the method includes obtaining a first standard curve, a second standard curve, and a reference standard curve. The first standard curve includes a first function relating starting number of copies of a first target nucleic acid to time-to-threshold. The second standard curve includes a second function relating starting number of copies of a second target nucleic acid to time-to-threshold. The reference standard curve includes a reference function relating starting number of copies of a reference nucleic acid to time-to-threshold. The first standard curve, the second standard curve, and the reference standard curve are generated prior to performing real-time quantitative isothermal amplification on the test sample. The method further includes obtaining the test sample from a mammalian subject. The test sample contains the first target nucleic acid, the second target nucleic acid, and the reference nucleic acid. The method further includes: adding the test sample to at least one reaction vessel containing a master mix for isothermal amplification of the first target nucleic acid, the second target nucleic acid, and the reference nucleic acid; starting at least one reaction of isothermal amplification in the at least one reaction vessel; determining a first tune-to-threshold value for the first target nucleic acid in the at least one reaction; determining a second time-to-threshold value for the second target nucleic acid in the at least one reaction; determining a reference time-to-threshold value for the reference nucleic acid in the at least one reaction; estimating a first starting number of copies of the first target nucleic acid in the test sample based on the first time-to-threshold value using the first function of the first standard curve; estimating a second starting number of copies of the second target nucleic acid in the test sample based on the second time-to-threshold value using the second function of the second standard curve; estimating a reference starting number of copies of the reference nucleic acid in the test sample based on the reference time-to-threshold value using the reference function provided by the reference standard curve; estimating a first relative abundance value of the first target nucleic acid in the test sample relative to the reference nucleic acid based on the first starting number of copies of the first target nucleic acid and the reference starting number of copies of the reference nucleic acid; estimating a second relative abundance value of the second target nucleic acid in the test sample relative to the reference nucleic acid based on the second starting number of copies of the second target nucleic acid and the reference starting number of copies of the reference nucleic acid; estimating the diagnostic score of the test sample based on the first relative abundance value of the first target nucleic acid and the second relative abundance value of the second target nucleic acid; and making a clinical diagnosis of a medical condition by comparing the diagnostic score of the test sample a predetermined threshold diagnostic score.

In another aspect, provided herein is an apparatus for estimating a diagnostic score of a test sample using real-time quantitative isothermal amplification. In some embodiments, the apparatus includes a first reaction vessel configured to hold a first aliquot of the test sample, and a second reaction vessel configured to bold a second aliquot of the test sample. The test sample contains a first target nucleic acid, a second target nucleic acid, and a reference nucleic acid. Each of the first target nucleic acid, the second target nucleic acid, and the reference nucleic acid comprises a mammalian host nucleic acid. Each of the first reaction vessel and the second reaction vessel contains a mister mix for isothermal amplification of the first target nucleic acid, the second target nucleic acid, and the reference nucleic acid. The first aliquot has a first volume. The second aliquot has a second volume greater than the first volume. The second target nucleic acid has a lower expected abundance than the first target nucleic acid in the test sample. The apparatus further includes a computer memory for storing a first threshold fluorescence intensity value and a second threshold fluorescence intensity value. The apparatus further includes means for starting a first isothermal amplification reaction in the first reaction vessel amplifying the first target nucleic acid and the reference nucleic acid. The first isothermal amplification reaction may produce first fluorescence associated with the first target nucleic acid and first reference fluorescence associated with the reference nucleic acid. The apparatus further includes a first fluorescence detector and a first reference fluorescence detector optically coupled to the first reaction vessel. The first fluorescence detector is configured to measure an intensity of the first fluorescence as a function of time and to measure a first reference intensity of the first reference fluorescence as a function of time. The apparatus further includes means for starting a second isothermal amplification reaction in the second reaction vessel amplifying the second target nucleic acid and the reference nucleic acid. The second isothermal amplification reaction may produce second fluorescence associated with the second target nucleic acid and second reference fluorescence associated with the reference nucleic acid. The apparatus further includes a second fluorescence detector and a second reference fluorescence detector optically coupled to the second reaction vessel. The second fluorescence detector is configured to measure an intensity of the second fluorescence as a function of time and to measure a second reference intensity of the second reference fluorescence as a function of time. The apparatus further includes a computer processor coupled to the first fluorescence detector, the second fluorescence detector, the first reference fluorescence detector, the second reference fluorescence detector, and the computer memory. The computer processor is configured to: determine a first time-to-threshold value for the first target nucleic acid based on the intensity of the first fluorescence as a function time and the first threshold fluorescence intensity value; determine a first reference time-to-threshold value for the reference nucleic acid based on the intensity of the first reference fluorescence as a function time and the first threshold fluorescence intensity value; estimate a first relative abundance value of the first target nucleic acid in the test sample relative to the reference nucleic acid based at least on the first time-to-threshold value and the first reference time-to-threshold value: determine a second time-to-threshold value for the second target nucleic acid based on the intensity of the second fluorescence as a function time and the second threshold fluorescence intensity value: determine a second reference time-to-threshold value for the reference nucleic acid based on the intensity of the second reference fluorescence as a function time and the second threshold fluorescence intensity value; estimate a second relative abundance value of the second target nucleic acid in the test sample relative to the reference nucleic acid based at least on the second time-to-threshold value and the second reference time-to-threshold value; and estimate the diagnostic score of the test sample based on the first relative abundance value for the first target nucleic acid and the second relative abundance value for the second target nucleic acid.

### BRIEF DESCRIPTION OF THE DRAWINGS

FIG. 1 shows an exemplary plot of fluorescence intensity as a function of time in a real-time quantitative isothermal amplification assay, according to some embodiments.

FIG. 2 shows exemplary plots of fluorescence intensities as a function of time in real-time quantitative isothermal amplification assays of eight samples with different concentrations of a target nucleic acid, according to some embodiments.

FIG. 3 shows a scatter plot of data points representing time-to-threshold values vs. logarithm of copy numbers obtained from the fluorescence curves shown in FIG. 2, and a straight line obtained from a linear regression on the data points, according to some embodiments.

FIG. 4 shows a simplified flowchart illustrating a method of estimating a diagnostic score by performing real-time quantitative isothermal amplification on a test sample according to some embodiments.

FIG. 5 shows a simplified flowchart illustrating a method of estimating a diagnostic score by performing real-time quantitative isothermal amplification of a test sample according to some other embodiments.

FIG. 6 shows a simplified flowchart illustrating a method of estimating a diagnostic score by peforming, real-time quantitative isothermal amplification on a test sample according to some embodiments.

FIG. 7 shows a schematic block diagram of an apparatus for estimating a relative abundance value using a real-time quantitative isothermal amplification assay according to some embodiments.

FIG. 8 is a graph showing a correlation plot with Pearson coefficient for relative abundance of a target nucleic acid (IFI27) to a reference nucleic acid (YWHAB) determined using a quantitative real time isothermal amplification assay as compared to a gold standard assay (NanoString nCounter). Values are plotted as Log₂(Fold Abundance) for both assays and assigned according to diagnosis: circles represent viral infection; squares represent bacterial sepsis; and triangles represent healthy control. The line represents a curve fit by standard linear regression analysis.

FIG. 9A shows the correlation plot with Pearson coefficient relating the diagnostic scores determined by the isothermal amplification assays and by the gold standard NanoString nCounter according to some embodiments.

FIG. 9B shows the HostDx-Fever score distribution based on qLAMP assays according to some embodiments.

FIG. 9C shows the HostDx-Fever score distribution based on NanoString nCounter according to some embodiments.

FIG. 10 illustrates an example in which the cutoff value is defined as the y-intercept of a linear regression fit to a standard curve titration according to some embodiments.

FIG. 11 shows the experimental results of qLAMP assays using multi-volume reaction vessels according to some embodiments.

### DETAILED DESCRIPTION

Before the present invention is described, it is to be understood that this invention is not limited to particular embodiments described, as such may, of course, vary. It is also to be understood that the terminology used herein is for the purpose of describing particular embodiments only, and is not intended to be limiting.

The disclosure provides methods, apparatuses, and kits for determining the relative abundance value of a target nucleic acid in a test sample. While qPCR is well known in the art, isothermal amplification techniques have failed to further advance methods of quantitating target nucleic acids in a clinically relevant sample. For example, qLAMP methods for quantifying bacterial or viral nucleic acids may not be reliable below 1,000 copies (see., Nixon et al., (2014), Bimolecular Detection and Quantitation 2: 4-10). Thus, what is needed is a method for quantifying the presence of a target mammalian mRNA in a sample, independent of the starting copy number of the target nucleic acid in the sample. Provided herein are methods, apparatuses and kits for determining the relative abundance of a target nucleic acid with respect to a reference nucleic acid in a test sample that is not predicated on the starting copy number and that does not require absolute quantitation of the target nucleic acid or the reference nucleic acid in the sample. The methods, apparatuses, and kits utilize real-time quantitative isothermal amplification to amplify target nucleic acids and reference nucleic acids in the test sample. In some aspects, the target nucleic acid is a mammalian host nucleic acid (e.g., mRNA) expressed by the host in response to a bacterial or viral infection. Relative quantitation is sufficient to allow for certain diagnostic algorithms that rely on multiple mammalian RNA markers (see, for example, Published Patent Applications WO 2016/145426; WO 2017/066641: WO 2018/004806; and WO 2017/214061) to be computed stably across samples without the need for standard curves to be run for each target in the same assay, or even without using standard curves.

In some embodiments, the quantitative real-time isothermal amplification comprises strand displacement amplification (SDA), transcription mediated amplification (TMA), nucleic acid sequence based amplification (NASBA), recombinase polymerase amplification (RPA), rolling circle amplification (RCA), ramification amplification, helicase-dependent isothermal DNA amplification (HDA), nicking enzyme amplification reaction (NEAR) and loop mediated isothermal amplification (LAMP) (see, e.g., Notomi et al., (2000) Nucleic Acids Research, 28(12)E63, incorporated herein by reference).

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications and accession numbers mentioned herein are incorporated herein by reference to disclose and describe the methods and/or materials in connection with which the publications are cited.

Generally, the nomenclature used herein and the laboratory procedures in cell culture, molecular biology, organic chemistry, and nucleic acid chemistry and hybridization described below are those well-known and commonly employed in the art. Standard techniques are used for nucleic acid and peptide synthesis. The techniques and procedures are generally performed according to conventional methods in the art and various general references (see generally, Sambrook et al. Molecular Cloning: A Laboratory Manual, 2d ed. 11989) Cold Spring Harbor Laboratory Press, Cold Spring Harbor, N.Y., which is incorporated herein by reference), which are provided throughout this document.

Unless defined otherwise, all technical and scientific terms used herein have the same meaning as is commonly understood by one of skill in the art to which the claimed subject matter belongs. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, the terms “a,” “an,” and “the” are defined to mean one or more and include the plural unless the context is inappropriate. In this application the use of the singular includes the plural unless specifically stated otherwise.

Ranges may be expressed herein as from “about” one specified value, and/or to “about” another specified value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. When such a range is expressed, another embodiment includes from the one specific value and/or to the other specified value. Similarly, when values are expressed as approximations, by use of the antecedent “about,” it will be understood that the specified value forms another embodiment. It will be further understood that the endpoints of each of the ranges are included with the range.

The word “or” as used herein means any one member of a particular list and also includes any combination of members of that list.

As used herein, the term “including” as well as other forms (e.g., “include”, “includes”, and “included”) is not limiting.

As used herein, the term “relative abundance value” refers to a measurement of target nucleic acids in a test sample. In some embodiments, a relative abundance value is estimated by performing isothermal amplification of a target nucleic acid and a housekeeping gene in a test sample, such that a difference in gene expression can be compared between different samples (e.g., samples from different subjects or samples obtained from a single subject at different times (e.g., pre- and post-treatments)).

As used herein, the term “target nucleic acid”, refers to a mammalian RNA sequence expressed from a mammalian host gene, for example in response to pathogenic (e.g., bacterial or viral) activity. The mammalian RNA can include human, and non-human mammalian animals such as equines, felines, canines, porcines and ovines. In a preferred embodiment, the target nucleic acid is mammalian mRNA. In some embodiments, the target nucleic acid encompasses a splice junction within the mammalian host mRNA.

As used herein, the term “reference nucleic acid” or “endogenous control” refers to a nucleic acid present in the test sample used to normalize the target nucleic acid quantities. The reference nucleic acid can include a native nucleic acid normally found in the test sample (e.g., a housekeeping gene mRNA) or can include a known amount of input material spiked into the test sample to normalize the target nucleic acid quantity.

As used herein, the term “test sample”, refers to a biological sample obtained from a mammal. In some instances, a biological sample is a clinical sample obtained during a clinical procedure or obtained by a physician or physician assistant, such as a cervical or vaginal swab, nasal swab, blood or blood component sample (e.g. plasma, serum, or PMBCs). In some embodiments, the biological sample includes an excreted biological sample such as mucus, stool, or urine sample. In some embodiments, the test sample is self-collected specimen, such as nasal swabs, cheek swabs, fingerstick blood, and the like.

The term “isothermal amplification”, refers to a process in Which a target nucleic acid is amplified using a constant, single, amplification temperature (e.g., froth about 30° C. to about 95° C.). Unlike standard PCR an isothermal amplification reaction does not include multiple cycles of denaturation, hybridization, and extension, of an annealed oligonucleotide to form a population of amplified target nucleic molecules (i.e., amplicons). There are various types of isothermal application known in the art, including but not limited to, loop-mediated isothermal amplification (LAMP), nucleic acid sequence based amplification NASBA, recombinase polymerase amplification (RPA), rolling circle amplification (RCA), nicking enzyme amplification reaction (NEAR), and helicase dependent amplification (HDA).

As used herein, the term “real-time quantitative isothermal amplification” refers to a process in which a target nucleic acid is amplified at a constant temperature and the target nucleic acid rate of amplification is monitored by fluorescence, rabidity, or similar measures (e.g., NEAR or LAMP). In some aspects, RNA (e.g., mRNA) is isolated from a biological sample and is used as a template to synthesize cDNA. Reverse-transcription is a well-known method for converting mRNA to cDNA. cDNA molecules are amplified under isothermal amplification conditions such that the production of amplified target nucleic acid can be detected and quantitated. As used herein, “amplification rate” is the rate at which a target nucleic acid amplicon is generated.

As used herein, a “polymerase” refers to one or more enzymes used together with strand displacement activity that performs template-directed synthesis of polynucleotides, e.g., DNA and/or RNA. The term encompasses both the full length polypeptide and a domain that has polymerase activity. Additional examples of commercially available polymerase enzymes include, but are not limited to: Klenow fragment (New England Biolabs® Inc.), Taq DNA polymerase (QIAGEN), 9 ° N™ DNA polymerase (New England Biolabs® Inc.), Deep Vent™ DNA polymerase (New England Biolabs® Inc.), Manta DNA polymerase (Enzymatics®), Bst DNA polymerase (New England Biolabs® Inc.), and phi29 DNA polymerase (New England Biolabs® Inc.). Polymerases such as Bst 2.0, Bst 3.0, and Bst 2.0 WarmStart® DNA polymerase (all commercially available from New England Biolabs® Inc.) are suitable for LAMP. LAMP is applicable to RNA upon use of reverse transcriptase (RTase) together with a DNA polymerase.

Polymerases include both DNA-dependent polymerases and RNA-dependent polymerases such as reverse transcriptase. At least five families Of DNA-dependent DNA polymerases are known, although most fall into families A, B and C. Other types of DNA polymerases include phage polymerases. Similarly, RNA polymerases typically include eukaryotic RNA polymerases I, II, and III, and bacterial RNA polymerases as well as phage and viral polymerases. RNA polymerases can be DNA-dependent and RNA-dependent.

As used herein, the term “standard curve” is given its plain and ordinary meaning in the art. Typically, a standard curve is derived from a set of samples of different concentrations of a target nucleic acid, for example by serial dilutions of a template. Time-to-threshold values are plotted against logarithm (e.g., base 10) of concentrations. Least square fit is used as the standard curve.

As used herein, the term “master mix for isothermal amplification” refers to a plurality of components such as dNTPs, salts (e.g., magnesium), and buffers required to perform an isothermal amplification assay. In some instances, the master mix for isothermal amplification is a real-time quantitative isothermal amplification master mix. In some embodiments, a master mix for isothermal amplification contains all of the components required for performing an isothermal amplification reaction except primers, probes, and nucleic acid template to be amplified.

The terms “fluorescent label” or “fluorophore” refer to a compound with a fluorescent emission maximum between about 400 and about 900 nm. These compounds with their emission maxima in nm in brackets include, but are not limited to, Syto-9, Syto-82, Cy2™ (506), GFP (Red Shined) (507), YO-PRO™-1 (509), YOY™-1 (509), Calcein (517), FITC (518), FluorX™ (519), Alexa™ (520), Rhodamine 110 (520), 5-FAM (522), Oregon Green™, 500 (522), Oregon Green™ 488 (524), RiboGreen™ (525), Rhodamine Green™ (527), Rhodamine 123 (529), Magnesium Green™ (531), Calcium Green™ (533), TO-PRO™-1 (533), TOTO®-1 (533), JOE (548), BODIPY® 530/550 (550), Di1 (565), BODIPY® 558/568 (568), BODIPY® 564/570 (570), Cy3™ (570), Alexa™ 546 (570), TRITC (572), Magnesium Orange™, (575), Phycanythrin R&B (575), Rhodamine Phalloidin (575), Calcium Orange™ (576), Pyronin Y (580), Rhodamine B (580), TAMRA (582), Rhodamine Red™ (590), Cy3.5™ (596), ROX (608), Calcium Crimson™ (615), Alexa™ 594 (615), Texas Red® (615), Nile Red (628), YO-PRO™-3 (631). YOYO™-3 (631), R-phycocyanin (642), C-Phycocyanin (648), TO-PRO™-3 (660), TOTO®-3 (660), DiD Di1C(5) (665), Cy5™ (670), Thiadicarbocyanine (671), and Cy5.5 (694). Additional fluorophores are disclosed in PCT Patent Publication No. WO 03/023357 and U.S. Pat. No. 7,671,218. Examples of these and other suitable fluorescent dye classes can be found in Hatgland et al., Handbook of Fluorescent Probes and Research Chemicals, Sixth Ed., Molecular Probes, Eugene, Ore. (1996); U.S. Pat. Nos. 3,194,805; 3,128,179; 5,187,288; 5,188,934: 5,227,487, 5,248,782; 5,304,645; 5,433,896; 5,442,045; 5,556959; 5,583,236; 5,808,044; 5,852,191; 5,986,086; 6,020,481; 6,162,931; 6,180,295; and 6,221,604; EP Patent No. 1408366; Smith et al., S. Chem. Soc. Perkin Trans. 2: 1195-1204 (1993); Whitaker, et al., Anal. Biochem. 207: 267-279 (1992): Krasoviskii and Bolotin, Organic Luminescent Materials, VCH Publishers, N Y. (1988); Zolliger, Color Chemistry, 2nd Edition, VCH Publishers, N.Y. (1991); Hirschberg et al., Biochemistry 37: 10381-10385 (1998); Fieser and Fieser, REAGENTS FOR ORGANIC SYNTHESIS, Volumes 1 to 17, Wiley, US (1995); and Geiger et al., Nature 359: 859-861 (1992).

There is extensive guidance in the art for selecting a quencher and fluorophore pair and their attachment to oligonucleotides (Haugland 1996; U.S. Pat. Nos. 3,996,345 and 4,351,760 and the like). Exemplary quenchers are described in U.S. Pat. No. 6,727.356, incorporated herein by reference. Other quenchers include, but are not limited to, bis-azo quenchers (U.S. Pat. No. 6,790,945) and dyes from Biosearch Technologies, Inc. (provided as Black Hole™ Quenchers: BH-1, BH-2 and BH-3 quenchers), Dabcyl, TAMRA and carboxy tetramethyl rhodamine.

Any suitable fluorophore may be used to fluorescently label a primer, probe, or nucleotide incorporated during an isothermal amplification reaction. In some embodiments, the fluorescent label is an intercalating agent, such as but not limited to, Syto-9, Syto-82, SYBR®, Hoechst 33258, or DAPI.

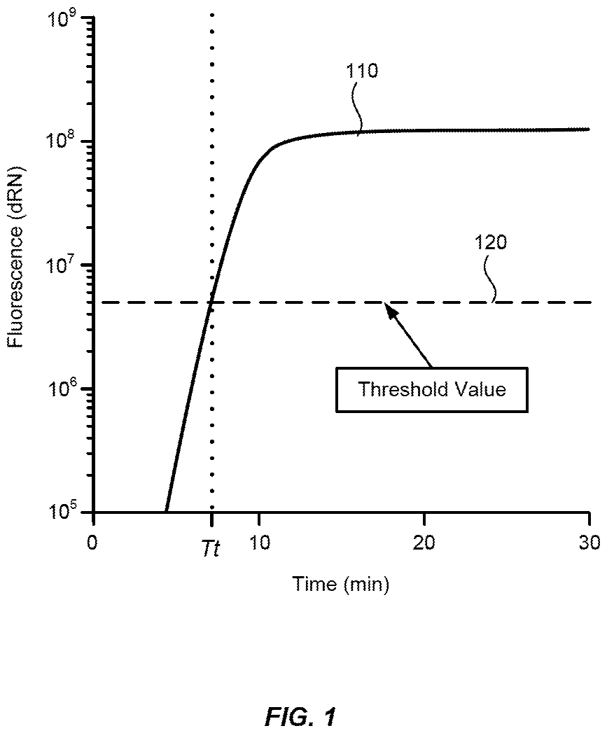

As used herein, the term “time-to-threshold” refers to the elapsed time from the moment when isothermal amplification is started to the moment when fluorescence intensity representing the concentration of the target nucleic acid reaches a pre-determined threshold value. In a plot of fluorescence intensity against time since isothermal amplification started (e.g., as illustrated in FIG. 1), the time-to-threshold is the X-axis crossing point where the fluorescence curve intercepts the horizontal line representing the threshold value.

As used herein, the term “number of copies” refers to the number of target nucleic acid molecules or reference nucleic acid molecules present in the original test sample (i.e., before real-time quantitative isothermal amplification).

As used herein, the tem' “N-fold serial dilution” refers to a stepwise dilution of a first component in solution (e.g., a reference nucleic acid) by a constant dilution factor (1:5; 1:10; 1:20; or 1:50) using an appropriate buffer (e.g., saline, water, etc.,). For example, a 10-fold serial dilution requires a stepwise dilution of a first component (e.g., reference nucleic acid present at a concentration of 100 mg/ml) by a factor of 10 (e.g., 1:10) to form a first serial dilution of the reference nucleic acid present at a concentration of 10 mg/ml; followed by a second 10-fold dilution of the first serial dilution (e.g., to form a second serial dilution of the reference nucleic acid present at a concentration of 1 mg/ml), and so on.

As used herein, the term “linear regression” refers to a statistical, linear approach to modeling the relationship between a dependent variable and one or more independent variables. In linear regression, the relationships are modeled using linear predictor functions whose unknown model parameters are estimated from the data. Such models are called linear models. Typically, for a single independent variable case, (x, y) data points are plotted in graphical form as a scatter plot, where x is the independent variable, and y is the dependent variable. Linear regression aims to obtain a “best fitting line” that represents the relationship between the dependent variable and the independent variable. Linear regression models are often fitted using the least squares approach, but they may also be fitted in other ways, such as by minimizing a “cost function” in some other norm (e.g., L1-norm penalty or L2-norm penalty).

As used herein, the term “linear dynamic range” of a quantitative isothermal amplification assay refers to the range of input concentrations of a target nucleic acid where a plot of time-to-threshold vs the logarithm (e.g., base 2 or base 10) of concentration is linear.

As used herein, the term “dynamic range” refers to the range (from maximum to minimum) of sample concentrations or input amounts that a given assay (e.g., LAMP) is capable of detecting.

As used herein, the term “diagnostic score” refers to an integrated score for classifying or diagnosing a medical condition. The diagnostic score combines levels of expression of several biomarkers identified as relevant to the medical condition using a certain algorithm into a single score to which a clinically relevant threshold can be applied. Exemplary algorithms are discussed in sections J, K, and L below.

As used herein, the term “population test” refers to a study of a clinically representative population for establishing statistics of expression levels of one or more biomarkers. The term “clinically representative population” used herein refers to a group of individuals that is large enough to establish statistical significance. For example, the population test may measure a biomarker across patients with and without a certain disease, and use a student's West, a Welch's t-test, a Mann-Whitney U test, or an analysis of variance (ANOVA), an F-test, or the like, to establish whether the biomarker is differentially expressed. The statistical significance may be set at, for example, p<0.05.

As used herein, the term “machine-learning model” refers to an algorithm or a statistical model that a computer system uses to perform a task relying on patterns and inference from a set of testing data. A certain algorithm that integrates several biomarkers into a diagnostic score may be a machine-learning model. Exemplary machine-learning models include linear regression, logistic regression, decision tree, support vector machine (SVM), random forest, K-means, artificial neural network (ANN), and the like.

As used herein, the term “hot start”, refers to a variation in the amplification process that prevents non-specification amplification of nucleic acids by inactivating the polymerase used for amplification. Hot-start reduces formation of primer dimers and non-specific annealing of primers to non-target nucleic acids that are subsequently extended during amplification. There are various techniques for a hot-start (see, e.g., Paul et al., (2010) Methods Mol Biol., 630: 301-18). For example, a polymerase can be inactivated through the use of antibodies, aptamers, or chemical modifications, so that the polymerase (which may be modestly active at room temperature and/or on ice) is blocked from functioning at sub-optimal temperatures. In the hot-start process, an initial activation step (e.g., at 95° C.) is performed to activate the polymerase. This initial heat-activation step also inactivates antibodies linked to the polymerase or removes chemical modifications from the polymerase (e.g.,. lysine modifications). Once these components are inactivated, the polymerase can amplify target nucleic acids present in the test sample. In some embodiments, the methods disclosed herein include a hot-start mechanism such as an antibody, aptamers or chemical modification to the polymerase used in the real-time quantitative isothermal amplification assay.

A “primer” refers to a polynucleotide sequence that hybridizes to a complementary Sequence on a target nucleic acid and serves as a point of initiation of nucleic acid synthesis. Primers can be of a variety of lengths and are often less than 80 nucleotides in length, for example 20-70 nucleotides, in length. In one embodiment, a full length target-specific primer of the instant application can comprise between 30 and 65 nucleotides in length, such as 30, 5, 40, 45, 50, 55, 60 or 65 nucleotides in length. In some embodiments, a multiplex amplification reaction mixture can include one or more full length target-specific primers having a length of between 20 and 65 nucleotides. Different length target-specific primers may be used in a multiplex amplification reaction such that one or more of the multiplex full length target-specific primers comprises a different nucleotide length as compared to the remainder of the multiplex full length target-specific primer pairs (e.g., a multiplex amplification reaction comprising a first full length target-specific primer having a nucleotide length of 45 nucleotides and a second full length target-specific primer having a nucleotide length of 58 nucleotides). The length and sequences of primers for use in isothermal amplification can be designed based on principles known to those of skill in the art, see, e.g., Innis et al., PCR Protocols: A Guide to Methods and Applications (Innis et al., eds, 1990). Various tools exist for primer design and evaluation (e.g., NCBI-Blast software). In the instant application, primers having highly degenerate sequences may be avoided to reduce the probability of mis-hybridization during the isothermal amplification reaction. Primers can be prepared from DNA, RNA, or a chimera of DNA and RNA portions. In some cases, a primer can include one or more modified nucleosides (e.g., 2-amino-deoxyadenosine) or non-natural nucleotide bases (e.g., uracil in a DNA primer). In some cases, a primer can include a fluorescent label such as a FRET donor and FRET acceptor moiety.

As used herein, the term “probe” refers to an oligonucleotide, whether occurring naturally or produced synthetically, recombinantly or by amplification, which is capable of hybridizing to another oligonucleotide of interest. A probe may be single-stranded or double-stranded. Probes are useful in the detection, identification and isolation of particular gene sequences. In one embodiment, it is contemplated that any probe used in the present invention will be labeled so that the label is detectable in any detection system including fluorescent, radioactive, and luminescent systems. It is not intended that the present invention is limited to any particular detection system or label.

As used herein, the tem “amplicon” retains its normal and customary use in the art. An amplicon is an amplification product generated from a DNA or cDNA template.

As used herein, the term “relative quantitation”, refers to comparison between an expression level of a target nucleic acid and an expression level of a reference nucleic acid in a single sample, or comparison between expression levels of a same target nucleic acid in different samples. Relative quantitation can be contrasted with “absolute quantitation” that involves determining the absolute quantity of target nucleic acids in a sample.

### A. Test Sample

In some embodiments, the methods, apparatuses, and related kits described herein utilize a test sample. In some embodiments, the test sample is obtained from a clinical sample (e.g., a nasal swab, biopsy, or blood draw), taken for diagnostic evaluation for the purpose of identifying a disease or a medical condition. In some embodiments, the test sample is obtained by a physician or veterinarian. In some embodiments, the test sample is self-collected specimen, such as nasal swabs, cheek swabs, and the like. In some embodiments, the test sample is obtained by a medical device (e.g., leukapheresis device). Any suitable test sample may be used to practice the invention.

In some embodiments, the sample is obtained from a mammal, such as but not limited to, a human. In some embodiments, the sample is obtained from a non-human mammal, such as but not limited to, a chimpanzee, cat, dog, pig, sheep, or cow. In some embodiments, the test sample is obtained from a healthy or diseased mammal. In some embodiments, the sample is obtained from a mammalian subject that is diagnosed with, or is suspected of having, a viral or bacterial infection. In some embodiments, the mammalian subject is suspected of having a bacterial infection (e.g., Streptococcus). In some embodiments, the mammalian subject is suspected of having a viral infection (e.g., HIV).

In some embodiments, the test sample obtained from the host comprises a bodily fluid such as urine, saliva, blood or a blood component, such as but not limited to, serum, plasma, and peripheral blood mononuclear cells (PMBCs). Any suitable bodily fluid may be used to practice the invention.

### B. Target Nucleic Acids

In some embodiments, the methods, apparatuses, and related kits described herein utilize target nucleic acids. In some embodiments, the target nucleic acids are mammalian nucleic acids. In some embodiments, the target nucleic acids are host nucleic acids (e.g., produced by a cell of a host or detected in a sample obtained from the source of the test sample). In some embodiments, the target nucleic acids are mammalian host nucleic acids (e.g., mammalian DNAs or RNAs). In some embodiments, the target nucleic acids are mammalian host RNAs. In a preferred embodiment, the target nucleic acids are mammalian host mRNAs. In one embodiment, the target nucleic acids encompass splice junctions within the mammalian host mRNAs. In one embodiment, the target nucleic acids encompass nucleic acids of pathogen origin detected simultaneously with mammalian host mRNAs.

In some embodiments, the target nucleic acids are present in a sample obtained from a mammalian host, and the target nucleic acids are isolated from the host sample (e.g., RNA extraction). In some embodiments, the target nucleic acids are present in a sample obtained from a mammalian host (e.g., whole blood), and the target nucleic acids are isolated from the host sample. (e.g., a QIAamp RNA blood mini kit, Qiagen, Catalog No: 52304).

In some embodiments, a mammalian host target nucleic acid is more abundant in the test sample as compared to a reference nucleic acid. In some embodiments, a mammalian host target nucleic acid is less abundant in the test sample as compared to the reference nucleic acid. Any suitable mammalian host nucleic acid may be used to practice the invention.

### C. Reference Nucleic Acid

In some embodiments, the methods, apparatuses, and related kits described herein utilize a reference nucleic acid. In some embodiments, the reference nucleic acid is a mammalian nucleic acid. In some embodiments, the reference nucleic acid is a host nucleic acid (e.g., produced by a cell of a host or detected in a sample obtained from the source of the test sample). In some embodiments, the reference nucleic acid is mammalian host nucleic acid (e.g., mammalian DNA or RNA). In some embodiments, the reference nucleic acid is a mammalian host RNA. In a preferred embodiment, the reference nucleic acid is a mammalian host mRNA. In some embodiments, the mammalian host nucleic acid is more abundant in the test sample as compared to a target nucleic acid. In some embodiments, the mammalian host nucleic acid is less abundant in the test sample as compared to a target nucleic acid. In a preferred embodiment, the reference nucleic acid and the target nucleic acids are expressed by different genes. In yet another embodiment, the reference nucleic acid and the target nucleic acid are expressed by different genes from the same mammalian organism (e.g., human or mouse). Any suitable reference nucleic acid may be used to practice the invention.

### Housekeeping Gene

In some embodiments, the reference nucleic acid is a housekeeping gene or a product thereof, such as a corresponding mRNA transcript. In some embodiments, the reference nucleic acid includes a mRNA transcript that is a pre-mRNA molecule, a 5′ capped mRNA molecule, a 3′ adenylated mRNA molecule, or a mature mRNA molecule. In a preferred embodiment, the reference nucleic acid is a mature mRNA molecule obtained from a mammalian host who is also the source of the test sample.

In some embodiments, the reference nucleic is a mammalian housekeeping gene or a gene product thereof (e.g., a mRNA). In some embodiments, the housekeeping gene is a gene that is constitutively expressed (i.e., continually transcribed) by one or more cell populations in the mammalian host. A constitutively expressed gene may be contrasted with a facultative gene, which refers to a gene that is transcribed only when needed. Exemplary housekeeping genes suitable for use with the invention include actin, GAPDH and ubiquitin. Any suitable housekeeping gene may be used to practice the invention.

In some embodiments, the housekeeping gene or product thereof is expressed at a relatively constant rate by a cell of the host, such that the expression rate of the house keeping gene can be used as a reference point against the expression of other host genes or gene products thereof.

In some embodiments, the reference nucleic acid is a human housekeeping gene. Exemplary human housekeeping genes suitable for use with the invention include, but are not limited to, KPNA6, RREB1, YWHAB, Chromosome 1 open reading frame 43 (Clorf43), Charged multivesicular body protein 2A (CHMP2A), ER membrane protein complex subunit 7 (EMC7), Glucose-6-phosphate isomerase (GPI), Proteasome subunit, beta type, 2 (PSMB2), Proteasome subunit, beta type, 4 (PSMB4), Member RAS oncogene family (RAB7A), Receptor accessory protein 5 (REEP5), small nuclear ribonucleoprotein D3 (SNRPD3), Valosin containing protein (VCP) and vacuolar protein sorting 29 homolog (VPS59). In some embodiments, any housekeeping gene provided at http://www/tau/ac/il˜eleis/HKG/ may be used (see, Eisenberg and Levanon., Trends Genet. (2013), 10: 569-74).

In some embodiments, the reference nucleic acid is a porcine housekeeping gene. Exemplary porcine housekeeping genes suitable for use with the intention include, but are not limited to, ACTB, B2M, GAPDH, HMBS, SDHA, HPRT1, TBP, YWHAZ and RPL32.

In some embodiments, the reference nucleic acid is a bovine housekeeping gene. Exemplary bovine housekeeping genes suitable for use with the invention include, but are not limited to, ACTB, GAPDH, HMBS, SF3A1 HPRT1, H2A, and SDHA.

In some embodiments, the reference nucleic acid is an equine housekeeping gene. Exemplary bovine housekeeping genes suitable for use with the invention include, but are not li mired to, ACTB, GAPDH TOP2B, KRT8 and RPS9.

### Test Sample Processing

### 1. Reaction Vessel

In some embodiments, a target nucleic acid and a reference nucleic acid from a test sample are provided in a first reaction vessel. As used herein, a ‘reaction vessel’ refers to a system in which the invention can be conducted including, but not limited to, test tubes, microcentrifuge tubes, wells, chambers, microwells (e.g., wells in a microliter plate, such as 96-, 384-, and 1536-well assay plates), capillary tubes, microfluidic devices, or a testing site on, or within, a suitable surface (including, but not limited to, glass, plastic, silicon, metal oxides, beads, and silanized (e.g., alkoxysilane) surfaces). Any suitable reaction vessel may be used to practice the invention.

In some embodiments, the reaction vessel contains less than 1000 μL of liquid during the quantitative isothermal amplification method. In another embodiment, the reaction vessel contains about 15 μL to about 750 μL of liquid during the quantitative isothermal amplification method.

In another embodiment, a target nucleic acid from a test sample is contained in a first reaction vessel and a reference nucleic acid from the test sample is contained in a second reaction vessel. The reaction vessel may be of any useful dimensions (e.g., width, length, height) and comprised of any suitable material. Preferably, the reaction vessel(s) of the instant application are in the micro- or nano-liter scale.

In some embodiments, multiple reaction vessels are used for multiple target nucleic acids, with each reaction vessel for isothermal amplification of a respective target nucleic acid.

In some embodiments, the reaction vessel can further comprise a capture region to isolate the target nucleic acid or reference nucleic acid from the test sample before, after, or during the quantitative isothermal amplification. In one embodiment, the reaction vessel can comprise a capture region to isolate the target nucleic acid and/or the reference nucleic acid from the test sample after the quantitative isothermal amplification. In some embodiments, the capture region can comprises filter, a matrix, a polymer, a gel, and a membrane (e.g., a silica membrane, a glass-fiber membrane, a cellulose membrane, a nitrocellulose membrane, a polysulfone membrane, a nylon membrane, a polyvinylidene difluoride membrane, a vinyl copolymer membrane, or an ion exchange membrane, including any described herein a fiber (e.g., a glass fiber), or a particle (e.g., a silica particle, a bead, an affinity resin, or an ion exchange resin).

Any suitable material may be used as the reaction vessel. The materials used to form a reaction vessel are selected with regard to physical and chemical characteristics that are desirable for proper functioning of the reaction vessel. Suitable, materials include polymeric materials, such as silicone polymers (e.g., polydimethylsiloxane and epoxy polymers), polyimides (e.g., commercially available Kapton® (poly(4,4′-oxydiphenylene-pyromellitimide, from DuPont, Wilmington, Del.) and Upilex™ (poly(biphenyl tetracarboxylic dianhydride), from Ube Industries, Ltd., Japan)), polycarbonates, polyesters, polyamides, polyesters, polyurethanes, polyfluorocarbons, fluorinated polymers (e.g., polyvinylfluoride, polyvinylidene fluoride, polytetrafluoroethylene, polychlorotrifluoroethylene, perfluoroalkoxy polymer, fluorinated ethylene-propylene, polyethylenetetrafluomethylene, polyethylenechlorotrifluoroethylene, perfluoropolyether, perfluorosulfonic acid, perfluoropolyoxetane, FFPM/FFKM (perfluorinated elastomer [perfluoroelastomer]), FPM/FKM (fluorocarbon [chlorotrifluoroethylenevinylidene fluoride]), as well as copolymers thereof), polyetheretherketones (PEEK), polystyrenes, polyacrylonitrile-butadiene-styrene)(ABS), acrylate and acrylic acid polymers such as polymethyl methacrylate, and other substituted and unsubstituted polyolefins (e.g. cycloolefin polymer, polypropylene, polybutylene, polyethylene (PE, e.g., cross-linked PE, high-density PE, medium-density PE, linear low-density PE, low-density PE, or ultra-high-molecular-weight PE), polymethylpentene, polybutene-1, polyisobutylene, ethylene propylene rubber, ethylene propylene diene monomer (M-class) rubber), and copolymers thereof (e.g., cycloolefin copolymer); ceramics, such as aluminum oxide, silicon oxide, zirconium oxide, and the like); semiconductors, such as silicon, gallium arsenide, and the like; glass: metals; as well as coated combinations, composites, and laminates thereof.

### 2. Isothermal Amplification Timing Mechanisms

### Hot Start

In some embodiments, the methods, apparatuses and kits provided herein Utilize various mechanisms to ensure amplification of the target nucleic acid and the reference nucleic acid can be reliably be compared (e.g., to one another and across various, non-sequential, experimental replicates). In some instances, the methods and apparatuses comprise a ‘hot-start’ mechanism that reduces or inhibits the production of non-specific (e.g., spurious) amplification products in the test sample. The hot start may ensure that each reaction starts at the same time. There are various techniques known in the art for performing hot-start amplification (see, e.g., Paul et al., (2010) Methods Mol Biol., 630: 301-18). Any suitable hot-start mechanism may be used to practice the invention.

In some embodiments, the ‘hot-start’ mechanism includes one or more antibodies that inactivate a polymerase of the quantitative isothermal amplification assay (e.g., a RNA polymerase or DNA polymerase).

In some embodiments, the ‘hot-start’ mechanism includes one or more aptamers that inactivate a polymerase of the quantitative isothermal amplification assay (see. e.g., WarmStart LAMP Kit, New England Biolabs Catalog No: E1700S; and WarmStart RTx Reverse Transcriptase, New England Biolabs Catalog No: M0380S). Nucleic-acid based aptamers suitable for use with a polymerase include those set forth in U.S. Pat. Nos.: 5,475,096; 5,670,637; 5,696,249; 5,874,557 and 5,693,502.

In some embodiments, the hot-start' mechanism includes one or more chemical modifications that inactivate a polymerase of the quantitative isothermal amplification assay. In some embodiments, the one or more chemical modifications include a lysine modification whereby one or more lysine residues present in the polymerase are chemically modified such that the polymerase is inactive until the amplification reaction reaches a temperature in excess of 90° C.

During a hot-start mechanism, heating is used to activate the polymerase. This initial heat-activation may also inactivate antibodies linked to the polymerase or remove chemical modifications from the polymerase (e.g., lysine modifications). Once these components are inactivated, the polymerase can amplify the target nucleic acids present in the test sample.

### Simultaneous Amplification

In some embodiments, the methods, apparatuses and kits provided herein utilize a simultaneous amplification step to ensure amplification of the target nucleic acid and the reference nucleic acid can be reliably be compared (e.g., to one another and across various, non-sequential, experimental replicates). In some embodiments, amplification of a target nucleic acid in the test sample (e.g., in a first reaction vessel) occurs simultaneously with amplification of a reference nucleic acid in the test sample (in a second reaction vessel). In some embodiments, the simultaneous amplification step includes amplification of a target nucleic acid and a reference nucleic acid in the same reaction vessel at the same time (e.g., identical start and stop amplification reaction times). In some embodiments, simultaneous amplification can include a ‘hot-start’ mechanism such that the amplification of the target nucleic acid and the reference nucleic acid (either in the same or different reaction vessels) does not begin until each of the target nucleic acids and the reference nucleic acids have reached the same designated temperature (e.g., 65° C.), at which time, the amplification reaction begins simultaneously.

### Asynchronous Amplification

In some embodiments, the methods, apparatuses and kits provided herein utilize an asynchronous amplification step of the target nucleic acid and the reference nucleic acid with a temporal indicator for the initiation of the amplification reactions so that two or more reactions can be reliably be compared (e.g., to one another and across various, non-sequential, experimental replicates). In some embodiments, amplification of a target nucleic acid in the test sample (e.g., in a first reaction vessel) occurs asynchronously as compared to amplification of a reference nucleic acid in the test sample (e.g., in a second reaction vessel). In some embodiments, asynchronous amplification includes amplification of a target nucleic acid and a reference nucleic acid in the same reaction vessel, for example, by providing primers and/or probes complementary to the target nucleic acid at Time 1, and providing primers and/or probes complementary to the reference nucleic acid at a later time, e.g., Time 2. In order to obtain a relative abundance value for the target nucleic acid in the test sample using the asynchronous amplification approach, it is important to determine the length of the amplification reaction for amplification of the target nucleic acid. Once the length of the amplification reaction is known, it can be used as the basis for performing amplification of the reference nucleic acid. As such, each of the target nucleic acid and the reference nucleic acid are amplified for the same period of time and under the same conditions, such that the output of the amplification reaction can be reliably compared to each other.

### E. Isothermal Amplification

Described herein are methods, apparatuses and related kits for isothermal amplification of a target nucleic acid. Isothermal amplification assay do not include cyclic heating and cooling steps as required by PCR. As such, isothermal amplification assays do not require expensive equipment such as thermocyclers to perform isothermal amplification (see, e.g., Gill and Ghaemi, (2008) Nucleos. Nucleot. Nucl., 27: 224-243: Kim and Easley (2011), Bioanalysis, 3: 227-239 and Yan et al., Mol. Biosyst., 10: 970-1003). In some embodiments, the isothermal amplification comprises a real-time isothermal amplification assay. In some embodiments, the isothermal amplification assay is a real-time quantitative isothermal amplification assay. Real-time isothermal amplification, also referred to as quantitative isothermal amplification, is often used to measure the quantity of an amplification product in real-time. Typically, quantitative isothermal amplification involves the use of a labeled probe (e.g., a fluorophore-containing probe or fluorescent dye) along with a set of standards in the amplification reaction, that allow for quantitation of the starting amount of target nucleic acids in the sample.

In some embodiments, the isothermal amplification comprises a reverse-transcriptase and a strand displacing isothermal enzyme, such as but not limited to, a Bst polymerase. Reverse-transcriptase isothermal amplification is a method used to synthesize and amplify DNA from RNA. Reverse transcriptase is an enzyme that reverse transcribes RNA into complementary DNA (cDNA), which is subsequently amplified by isothermal amplification. Reverse-transcriptase isothermal amplification can be used in gene expression profiling, to determine the expression of a gene or to identify the sequence of an RNA transcript, including transcription start and termination sites.

In some embodiments, the isothermal amplification comprises Strand Displacement Amplification (SDA). SDA relies on the ability of certain restriction enzymes to nick DNA and the ability of a 5′-3′ exonuclease-deficient polymerase to extend and displace the downstream strand. Exponential nucleic acid amplification can be achieved by coupling sense and antisense reactions in which strand displacement from the sense reaction serves as a template for the antisense reaction. For example, nickase enzymes which do not cut DNA but produce a nick on one of the DNA strands, such as Nt.Alwl can be used. SDA can also include a combination of a heat-stable restriction enzyme (e.g., Aval) and a heat-stable polymerase (e.g., Bst polymerase). Such combinations are known to increase amplification efficiency by about 10-fold, thus enabling amplification of target nucleic acids present as a single copy in the test sample.

In some embodiments, the amplification reaction assay comprises Transcription Mediated Amplification (TMA) or Nucleic Acid Sequence Based Amplification (NASBA). In TMA and NASBA, a RNA polymerase is used to amplify RNA sequences. Generally, the methods utilize a first and second primer and two or three enzymes (e.g., RNA polymerase, reverse transcriptase and optionally RNase H) and a first primer having a promoter sequence for the RNA polymerase. In the first step of target nucleic acid amplification, the first primer hybridizes to a target RNA (e.g., ribosomal RNA) at a defined site. Reverse transcriptase creates a cDNA copy of the target rRNA by extension from the 3′ end of the promoter primer. The RNA in the resulting RNA:DNA duplex can be degraded by the RNase activity of the reverse transcriptase if present or the additional RNase H. Next, a second primer binds to the cDNA copy and a new strand of DNA is synthesized from the end of this primer by reverse transcriptase, creating a double-stranded DNA molecule. RNA polymerase recognizes the promoter sequence in the DNA template and initiates transcription. Each of the newly synthesized RNA amplicons re-enters the above process and serves as a template for a new rotund of replication.

In some embodiments, the amplification reaction comprises Recombinase Polymerase Amplification (RPA). As such, isothermal amplification of the target nucleic acid is achieved by the binding of opposing oligonucleotide primers to the template nucleic acid and extension of the primers by a polymerase (see, e.g., Piepenburg et al., (2006) PloS Biol., 4(7): e204). In some instances, the isothermal amplification reaction includes a recombinase (e.g., RecA from bacteria or UvsX from bacteriophage T4), one or more cofactors (e.g., ATP or UvsY), and/or one or more single stranded binding proteins (SSB).

In some embodiments, the isothermal amplification assay comprises Helicase Dependent Amplification (HDA). HDA mimics an in vivo system that uses a DNA helicase enzyme to generate single-stranded templates for primer hybridization and subsequent primer extension by a DNA polymerase. In a first step of the HDA reaction, a helicase enzyme traverses along a target nucleic acid creating a single-stranded target region that allows primers to anneal to the target region. A DNA polymerase then extends the 3′ end of each primer using free deoxyribonucleoside triphosphates (dNTPs) to produce two DNA replicates. The two replicated DNA strands independently enter the next cycle of HDA, resulting in exponential nucleic acid amplification of the target nucleic acid.

In some embodiments, the isothermal amplification assay comprises Rolling Circle Amplification (RCA). Typically in RCA, a polymerase extends a primer continuously around a circular template, generating a long amplification product that contains many repeated copies of the circular template. By the end of the reaction, the polymerase has generated thousands of copies of the circular template, with the chain of copies tethered to the original target nucleic acid. RCA allows for spatial resolution of target nucleic acids and rapid nucleic acid amplification. Ramification amplification is a variation of RCA and utilizes a closed circular probe (C-probe) or padlock probe and a polymerase with a high processivity to exponentially amplify the C-probe under isothermal conditions.

In some embodiments, the isothermal amplification assay comprises Loop-Mediated Isothermal Amplification (LAMP). LAMP oilers selectivity and employs a polymerase and a set of specially designed primers that recognize distinct sequences in the target nucleic acid (see, e.g., Nixon et al., (2014) Bimolecular Detection and Quantitation, 2: 4-10; Schuler et al., (2016) Anal Methods., 8: 2750-2755; and Schoepp et al., (2017) Sci. Transl. Med., 9: eaa13693). Unlike PCR, the target nucleic acid is amplified at a constant temperature (e.g., 60-65° C.) using multiple inner and outer primers and a polymerase having strand displacement activity. In some instances, an inner primer pair containing a nucleic acid sequence complementary to a portion of the sense and antisense strands of the target nucleic acid initiate LAMP. Following strand displacement synthesis by the inner primers, strand displacement synthesis primed by an outer primer pair can cause release of a single-stranded amplicon. The single-stranded amplicon may serve as a template for further synthesis primed by a second inner and second outer primer that hybridize to the other end of the target nucleic acid and produce a stem-loop nucleic acid structure. In subsequent LAMP cycling, one inner primer hybridizes to the loop on the product and initiates displacement and target nucleic acid synthesis, yielding the original stem-loop product and a new stein-loop product with a stem twice as long. Additionally, the 3′ terminus of an amplicon loop structure serves as initiation site for self-templating strand synthesis, yielding a hairpin-like amplicon that forms an additional loop structure to prime subsequent rounds of self-templated amplification. The amplification continues with accumulation of many copies of the target nucleic acid. The final products of the LAMP process are stem-loop nucleic acids with concatenated repeats of the target nucleic acid in cauliflower-like structures with multiple loops formed by annealing between alternately inverted repeats of a target nucleic acid sequence in the same strand.

In some embodiments, the isothermal amplification assay comprises a digital reverse-transcription loop-mediated isothermal amplification (dRT-LAMP) reaction for quantifying the target nucleic acid (see. e.g., Khorosheva et al., (2016) Nucleic Acid Research, 44: 2 e10. Typically, LAMP assays produce a detectable signal (e.g., fluorescence) during the amplification reaction. In some embodiments, fluorescence can be detected anti quantified. Any suitable method for detecting and quantifying florescence can be used. In some instances, a device such as Applied Biosystem's QuantStudio can be used to detect and quantify fluorescence from the isothermal amplification assay.

### F. Detection

Any suitable method for detecting amplification of a target nucleic acid in a test sample by quantitative real-time isothermal amplification may be used to practice the invention. In some embodiments, quantitative real-time isothermal amplification of a target nucleic acid in a test sample can be determined by detecting of one or more different (distinct) fluorescent labels attached to nucleotides or nucleotide analogs incorporated during isothermal amplification of the target nucleic acid (e.g., 5-FAM (522 nm), ROX (608 nm), FITC (518 nm) and Nile Red (628 nm). In another embodiment, quantitative real-time isothermal amplification of a target nucleic acid in a test sample can be determined by detection of a single fluorophore species (e.g., ROX (608 nm)) attached to nucleotides or nucleotide analogs incorporated during isothermal amplification of the target nucleic acid. In some embodiments, each fluorophore species used emits a fluorescent signal that is distinct from any other fluorophore species, such that each fluorophore can be readily detected among other fluorophore species present in the assay.

In some embodiments, methods of detecting amplification of a target nucleic acid in a test sample by quantitative real-time isothermal amplification can include using intercalating fluorescent dyes, such as SYTO dyes (SYTO 9 or SYTO 82).

In some embodiments, methods of detecting amplification of a target nucleic acid in a test sample by quantitative real-time isothermal amplification can include using unlabeled primers to isothermally amplify the target nucleic acid in the test sample, and a labeled probe (e.g., having a fluorophore) to detect isothermal amplification of the target nucleic acid in the test sample.

In some embodiments, methods of detecting amplification of a target nucleic acid in a test sample by quantitative real-time isothermal amplification can include using unlabeled primers to isothermally amplify a target nucleic acid present in the test sample, and a probe having a 5-FAM dye label on the 5′ end and a minor groove binder (MGB) and non-fluorescent quencher on the 3′ end to detect isothermal amplification of the target nucleic acid (e.g., TaqMan Gene Expression Assays from ThermoFisher Scientific).

In some embodiments, detecting amplification of the target nucleic acid in the test sample can be performed using a one-step, or two-step, quantitative real-time isothermal amplification assay. In a one-step quantitative real-time isothermal amplification assay, reverse transcription is combined with quantitative isothermal amplification to form a single quantitative real-time isothermal amplification assay. A one-step assay reduces the number of hands-on manipulations as well as the total time to process a test sample. In some instances, for example, when some of the cDNA from a reverse transcription reaction is needed for other reactions or is to be retained, the quantitative real-time isothermal amplification assay can comprise a two-step assay. A two-step assay comprises a first-step, where reverse transcription is performed, followed by a second-step, where quantitative isothermal amplification is performed. It is within the scope of the skilled artisan to determine whether a one-step or two-step assay should be performed.

### G. Generating Standard Curves

Because isothermal amplification assays may amplify different nucleic acids with different efficiencies, it may be necessary to calibrate time-to-threshold values measured in a real-time isothermal amplification assay to absolute copy numbers of a target nucleic acid and a reference nucleic acid. This may be accomplished by establishing standard curves for the isothermal amplifications of the target nucleic acid and the reference nucleic acid. The standard curves can be obtained by performing real-time isothermal amplification assays using quantitated calibrator samples with multiple blown input concentrations.

In some embodiments, to generate a standard curve, quantitated calibrator samples are obtained by performing serial dilutions of a quantitated material. As an example, a template with a concentration of approximately 1×10⁹ copies/μL of the target nucleic acid is prepared. To obtain calibrator samples with different input concentrations, the template is serially diluted in a buffer at 10-fold concentration intervals yielding templates covering a range of concentrations from approximately 10⁹ copies/μL to approximately 10² copies/μL. For example, eight calibrator samples are obtained at concentrations of approximately 1×10⁹, 1×10⁸, 1×10⁷, 1×10⁶, 1×10⁵, 1×10⁴, 1×10³, and 1×10² copies/μL, respectively. The precise concentration of each calibrator sample can be determined using methods known in the art.

To obtain a standard curve, a real-time isothermal amplification assay is performed for each aliquot with a blown quantity (e.g., 1 μL) of a respective calibrator sample with a respective concentration of the target nucleic acid. For example, each aliquot may include 1 μL of the respective calibrator sample. For instance, in the above example where there are eight calibrator samples covering a range of concentrations from 10⁹ copies/μL to 10² copies/μL, eight aliquots of the eight calibrator samples contain the following number of copies of the target nucleic acid, respectively: Copy Number₁=1×10⁹, CopyNumber₂=1×10⁸, CopyNumber₃=1×10⁷, CopyNumber₄=1×10⁶, CopyNumber₅=1×10⁵, CopyNumber₆=1×10⁴, CopyNumber₇=1×10³, CopyNumber₈=1×10².

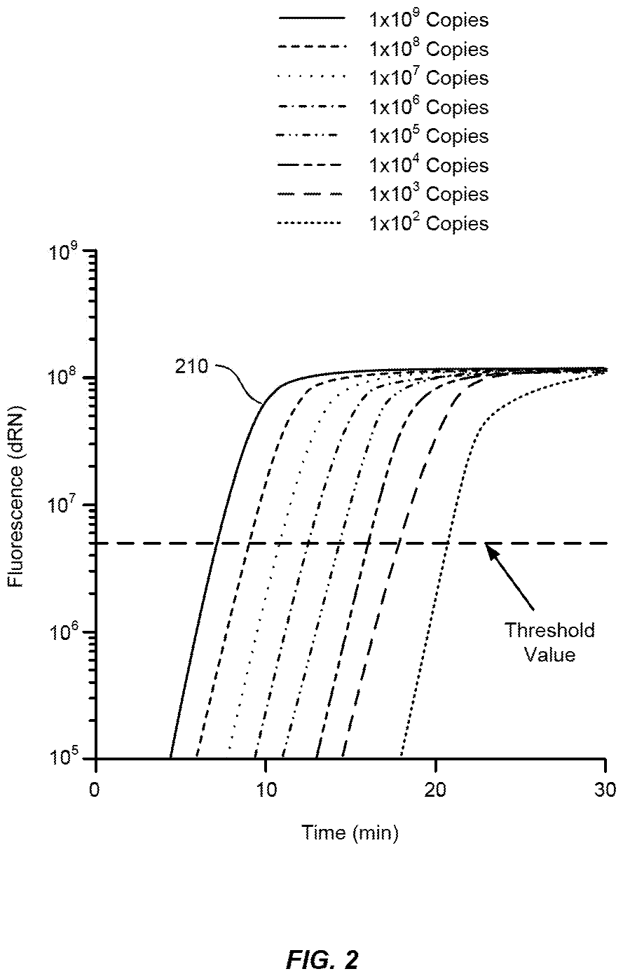

In a real-time isothermal amplification assay for each respective calibrator sample, the intensity of the fluorescence emitted by intercalating fluorescent dyes (e.g., dsDNA dyes) or fluorescent labels for the target nucleic acid is measured as a function of time. FIG. 1 shows an exemplary plot 110 of fluorescence intensity as a function of time in a real-time quantitative isothermal amplification assay according to some embodiments. The dashed line 120 represents a pre-determined threshold intensity. The elapsed time from the moment when isothermal amplification is started is the time-to-threshold Tt. FIG. 2 shows exemplary plots 210 of fluorescence intensities as a function of time in real-time quantitative isothermal amplification assays of eight samples with different concentrations of a target nucleic acid, according to some embodiments. A respective time-to-threshold value can be determined from each respective fluorescence curve 210 as a function of time. Thus, eight time-to-threshold values Tt₁, Tt₂, Tt₅, T₄, T₅, Tt₆, Tt₇, Tt₈, are obtained for the eight calibrator samples, respectively.

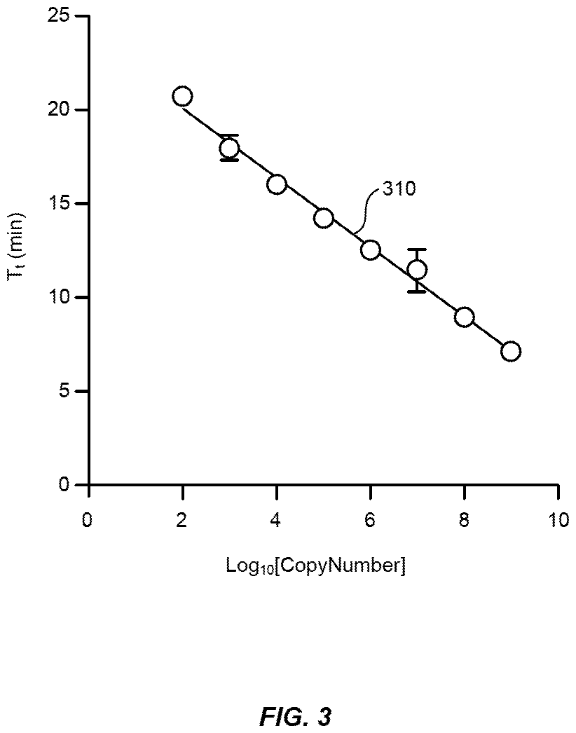

For exponential amplifications, the time-to-threshold is linearly proportional to the logarithm (e.g., logarithm to base 10) of the staring copy number (also referred to as template abundance). FIG. 3 shows a scatter plot of data points (represented by the circles), obtained from the fluorescence curves shown in FIG. 2, according to some embodiments. Each data. point represents a data pair [Log₁₀(CopyNumber), Tt] (note that CopyNumber refers to starting number of copies of a nucleic acid in an isothermal amplification assay). As illustrated, the data points fall approximately on a straight line 310. A linear regression is performed on the data points in the plot to obtain the straight line 310 that best fits the data points with the least amount of total deviations. The result of the linear regression is a straight lure represented by the following equation,

Tt=m×Log₁₀(CopyNumber)+b, (1)

Where m is the slope of the line 310, and b is y-intercept. The slope in represents the efficiency of the isothermal amplification of the target nucleic acid; b represents a time-to-threshold as template copy number approaches zero. The straight line represented by Equation (1) is referred to as the standard curve.

In some embodiments, replicates (e.g., triplicates) of isothermal amplification assays may be run for each sample in order to gain a higher level of confidence in the data. Replicate time-to-threshold values can be averaged, and standard deviations can be calculated.

Once the standard curve is established for a given isothermal amplification assay, the standard curve can be used to convert a time-to-threshold value to a starting copy number for future runs of the isothermal amplification assay of unknown starting numbers of copies of the target nucleic acid, using the following equation,

$$CopyNumber = {10}^{\frac{Tt - b}{m}} . \qquad ( 2 )$$

### Dynamic Range of a Standard Curve

Normally, the data points at every low copy numbers or very high copy numbers may fall off of the straight line 310. The range of copy numbers within which the data points can be represented by the straight line 310 is referred to as the dynamic range of the standard curve. The linear relationship between the time-to-threshold and the logarithmic of copy number represented by the standard curve would be valid only within the dynamic range.

If the amplification efficiencies for a target nucleic acid and a reference nucleic acid are different for a given isothermal amplification assay, it may be necessary to obtain separate standard curves for the target nucleic acid and the reference nucleic acid. Thus, two sets of real-time isothermal amplification assays may be performed, one set for establishing the standard curve for the target nucleic acid, the other set for establishing the standard curve for the reference nucleic acid. In cases where multiple target nucleic acids are considered, a standard curve for each target nucleic acid may be obtained.

### Off-Board Standard Curves

In some embodiments, the standard curves are generated prior to obtaining a test sample. That is, the standard curves are not generated on-board with the quantitative isothermal amplification of the test sample. Such standard curves may be referred to as off-board standard curves. Off-board standard curves may be used for estimating relative abundance values, as described below.

### H. Estimating Relative Abundance Values

For a test sample of unknown input concentration of a target nucleic acid A, a first real-time isothermal amplification assay is performed for a first aliquot of the test sample to obtain a first time-to-threshold value Tt_{A} with respect to the target nucleic acid A. A second real-time isothermal amplification assay is performed for a second aliquot of the test sample to obtain a second time-to-threshold value Tt_{B} with respect to a reference nucleic acid B. The first aliquot and the second aliquot contain substantially the same amount of the test sample. The first time-to-threshold value Tt_{A} may be converted into starting number of copies of the target nucleic acid A using the standard curve of the target nucleic acid A:

$${CopyNumber}_{A} = {10}^{\frac{T {t}_{A} - b}{m}} . \qquad ( 3 )$$

The second time-to-threshold value Tt_{B} may be convened into starting number of copies of the reference nucleic acid B using the standard curve of the reference nucleic acid B:

$${CopyNumber}_{B} = {10}^{\frac{{Tt}_{B} - b}{m}} . \qquad ( 4 )$$

The starting number of copies of the target nucleic acid A is normalized against that of the reference nucleic acid B to obtain a relative abundance value as.

$$Relative Abundance ( A / B ) = \frac{{CopyNumber}_{A}}{{CopyNumber}_{B}} . \qquad ( 5 )$$

For example, if the starting number of copies of the target nucleic acid A is 1000, and the starting number of copies of the reference nucleic acid B is 10, the relative abundance (A/B) can be calculated as,

$$Relative Abundance ( A / B ) = \frac{{CopyNumber}_{A}}{{CopyNumber}_{B}} = \frac{1000}{10} = 100.$$

In cases where the first aliquot and the second aliquot contain different amounts of the test sample, relative abundance is obtained as,

$$Relative Abundance ( A / B ) = \frac{{CopyNumber}_{A}}{{CopyNumber}_{B}} \cdot \frac{{M}_{2}}{{M}_{1}} , \qquad ( 6 )$$

where M₁ and M₂ are the masses (or volume) of the first aliquot and the second aliquot, respectively.

For example, if M₁=1, M₂=2, the starting number of copies of the target nucleic acid CopyNumber_{A} is 1000, and the starting number of copies of the reference nucleic acid CopyNumber_{B} is 10, the relative abundance (A/B) can be calculated as,

$$Relative Abundance ( A / B ) = \frac{{CopyNumber}_{A}}{{CopyNumber}_{B}} \cdot \frac{{M}_{2}}{{M}_{1}} = \frac{1000 \times 2}{10 \times 1} = 200.$$

In some other embodiments, one isothermal amplification assay may be performed for both the target nucleic acid A and the reference nucleic acid B. Because the isothermal amplification assay is performed on the same aliquot of the test sample, Equation (5) can be used to obtain the relative abundance.

### Relative Abundance Without Standard Curves

In cases where the amplification efficiencies for a target nucleic acid and a reference nucleic acid have approximately the same value that is known, and the expected copy number for the target nucleic acid and the expected copy number for the reference nucleic acid are within the dynamic range, relative abundance may be obtained directly from time-to-threshold values without using standard curves. For example, assuming that the amplification efficiency for both the target nucleic acid and the reference nucleic acid is in, and that b is identical for both assays as a consequence of the similar efficiencies, the relative abundance may be calculated as,

$$Relative Abundance ( A / B ) = {10}^{\frac{{Tt}_{A} - {Tt}_{B}}{m}} . \qquad ( 7 )$$

As described above, in cases where the amplification efficiencies for a target nucleic acid and a reference nucleic acid have approximately the same value that is blown, determining the relative abundance of a target nucleic acid in a test sample is not predicated on the starting copy number of the target nucleic acid, and does not require absolute quantitation of the target nucleic acid in the sample.

### I. Multiple Target Nucleic Acids

In some embodiments, a relative abundance value for a target nucleic acid can be used in combination with one or more additional relative abundance values for additional target nucleic acids in the test sample. In some embodiments, a plurality of relative abundance values for a plurality of target nucleic acids in a test sample can be used as inputs to an algorithm that can output a single diagnostic score, such as one that can discriminate between viral and bacterial infections in the test sample and/or diagnose the test sample as having a bacterial and/or viral infection (see, e.g., Sweeney et M., (2016), Sci. Transl. Med., 8: 346ras91346ra91).

In some embodiments, target nucleic acids selected for evaluation are selected from target nucleic acids known to have increased host expression as a result of bacterial infection, such as CTSB, TNIP1, GPAA1, and HK3. In some embodiments, target nucleic acids selected for evaluation are selected from target nucleic acids known to have increased host expression as a result of viral infection, such as IFI27, JUP, and LAX1.

In some embodiments, genes are combined using a machine learning or other algorithm to produce a single diagnostic score, such as one that can differentiate between bacterial and viral infections. Such a score may be higher in patients with bacterial infections and lower in patients with viral infections, such that a physician seeing a patient with a diagnostic score above a set threshold is directed to an action of treatment with antibiotics. In these embodiments, the algorithmic score carries more diagnostic power than any individual gene level alone has a greater area under the receiver-operating-characteristic curve for discrimination of bacterial from viral infections).

In some embodiments, types of algorithms for integrating multiple biomarkers into a single diagnostic score may include, but not limited to, a difference of geometric means, a difference of arithmetic means, a difference of sums, a simple sum, and the like. In some embodiments, a diagnostic score may be estimated based on the relative abundance values of multiple biomarkers using machine-learning models, such as a regression model, a tree-based machine-learning model, a support vector machine (SVM) model, an artificial neural network (ANN) model, or the like.

### J. Method For Estimating A Diagnostic Score By Performing Real-Time Quantitative Isothermal Amplification Using Off-board Standard Curves

Absolute quantification usually requires that the standard curves are generated in the same assay as the test sample to ensure proper calibration and adequate precision. The standard curves so obtained are referred to as on-board stand curves. According to some embodiments, a method of estimating a diagnostic score using real-time quantitative. isothermal amplification of multiple biomarkers only require relative quantification, and therefore off-board standard curves may be used. This may enable a new class of ultrafast diagnostic real-time quantitative isothermal amplification assays that are capable of making accurate diagnoses reliably and economically.

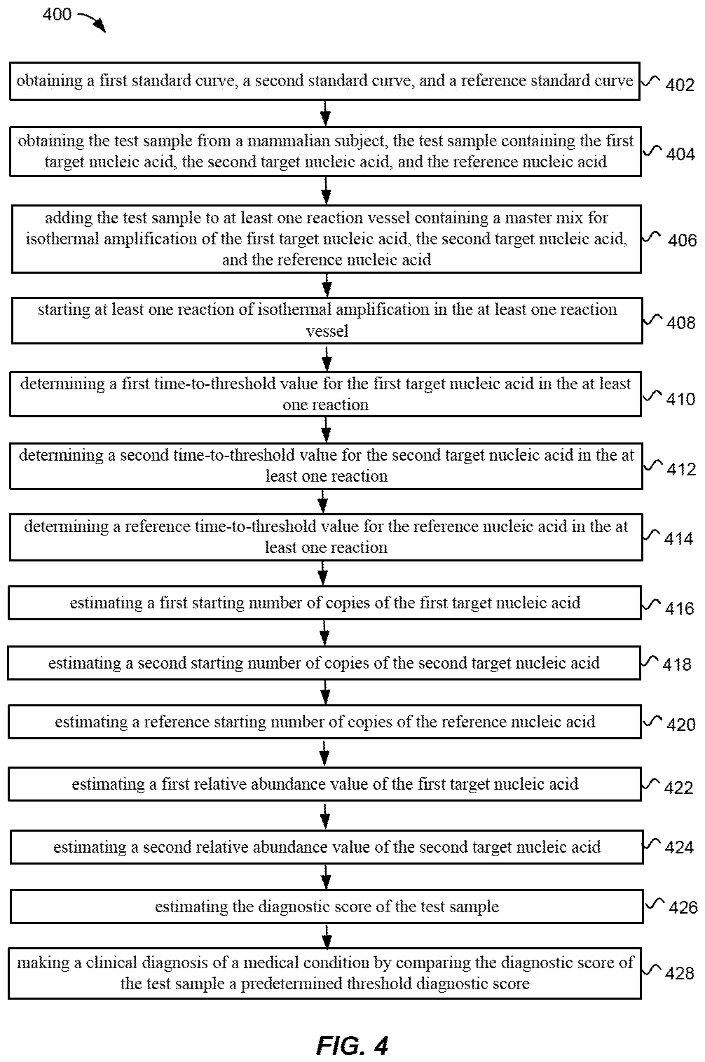

FIG. 4 shows a simplified flowchart illustrating a method of estimating a diagnostic score by performing real-time quantitative isothermal amplification on a test sample according to some embodiments.

At 402, a first standard curve, a second standard curve, and a reference standard curve are obtained. The first standard curve includes a first function relating starting number of copies of a first target nucleic acid to time-to-threshold. The second standard curve includes a second function relating starting number of copies of a second target nucleic acid to time-to-threshold. The reference standard curve includes a reference function relating starting number of copies of a reference nucleic acid to time-to-threshold. The first standard curve, the second standard curve, and the reference standard curve are generated prior to performing real-time quantitative isothermal amplification on the test sample.

At 404, the test sample is obtained from a manmialian subject. The test sample contains the first target nucleic acid, the second target nucleic acid, and the reference nucleic acid.

At 406, the test sample is added to at least one reaction vessel. The at least one reaction vessel contains a master mix for isothermal amplification of the first target nucleic acid, the second target nucleic acid, and the reference nucleic acid.

At 408, at least one reaction of isothermal amplification is started in the at least one reaction vessel.

At 410, a first time-to-threshold value is determined for the first target nucleic acid in the at least one reaction.

At 412, a second time-to-threshold value is determined for the second target nucleic acid in the at least one reaction.

At 414, a reference time-to-threshold value is determined for the reference nucleic acid in the at least one reaction.

At 416, a first starting number of copies of the first target nucleic acid in the test sample is estimated based on the first time-to-threshold value using the first function of the first standard curve.

At 418, a second starting number of copies of the second target nucleic acid in the test sample is estimated based on the second time-to-threshold value using the second function of the second standard curve.

At 420, a reference starting number of copies of the reference nucleic acid in the test sample is estimated based on the reference time-to-threshold value using the reference function provided by the reference standard curve.

At 422, a first relative abundance value of the first target nucleic acid in the test sample relative to the reference nucleic acid is estimated based on the first starting number of copies of the first target nucleic acid and the reference starting number of copies of the reference nucleic acid.

At 424, a second relative abundance value of the second target nucleic acid in the test sample relative to the reference nucleic acid is estimated based on the second starting number of copies of the second target nucleic acid and the reference starting number of copies of the reference nucleic acid.

At 426, the diagnostic score of the test sample is estimated based on the first relative Abundance value of the first target nucleic acid and the second relative abundance value of the second target nucleic acid.

At 428, a clinical diagnosis of a medical condition is made by comparing the diagnostic score of the test sample a predetermined threshold diagnostic score.

In some embodiments, the isothermal amplification is loop-mediated isothermal amplification (LAMP).

In some embodiments, the at least one reaction of isothermal amplification in the at least one reaction vessel is started using a hot start mechanism.

In some embodiments, the diagnostic score relates to a difference between the first time-to-threshold value for the first target nucleic acid and the second time-to-threshold value for the second target nucleic acid.

It should be appreciated that the specific steps illustrated in FIG. 4 provide a particular method of estimating a diagnostic score by performing real-time quantitative isothermal amplification according to some embodiments of the present invention. Other sequences of steps may also be performed according to alternative embodiments. For example, alternative embodiments of the present invention may perform the steps outlined above in a different order. Moreover, the individual steps illustrated in FIG. 4 may include multiple sub-steps that may be performed in various sequences as appropriate to the individual step. Furthermore, additional steps may be added or removed depending on the particular applications. One of ordinary skill in the art would recognize many variations, modifications, and alternatives.

### K. Method For Estimating A Diagnostic Score By Performing Real-Time Quantitative Isothermal Amplification Without Using Standard Curves

According to some embodiments, a method of estimating a diagnostic score may utilize real-time quantitative isothermal amplification without using standard curves. For example, a diagnostic score may integrate expression levels of multiple previously identified biomarkers. If it has been pre-verified over a target test population that isothermal amplification assays of the identified biomarkers are expected to perform within the linear dynamic range, the time-to-threshold values may be directly plug into an algorithm for estimating the diagnostic score without being converted into copy numbers using standard curves. In some embodiments, a clinically relevant threshold diagnostic score may be established by a population study. In the population study, real-time quantitative isothermal amplification assays are performed across a clinical cohort of patients of interest. The time-to-threshold values obtained from the real-time quantitative isothermal amplification assays are used to train a statistical model to establish the threshold diagnostic score. Once established, the threshold diagnostic score can be used to diagnose patients.

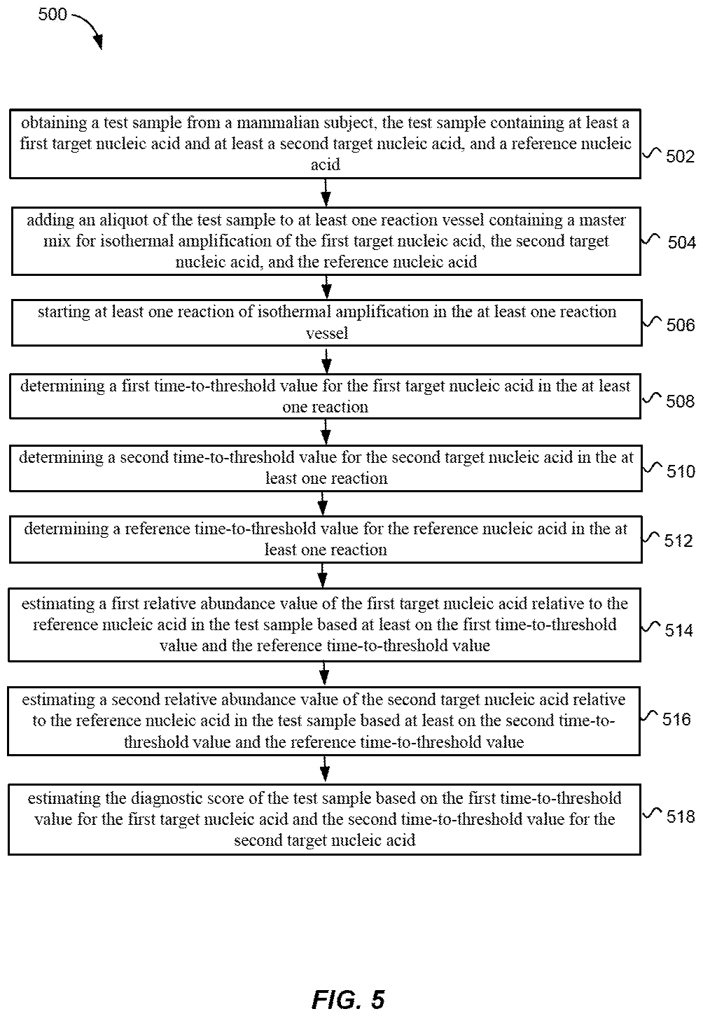

FIG. 5 shows a simplified flowchart illustrating a method of estimating a diagnostic score by performing real-time quantitative isothermal amplification of a test sample without using standard curves according to some other embodiments.

At 502, a test sample is obtained from a mammalian subject. The test sample contains at least a first target nucleic acid and at least a second target nucleic acid, and a reference nucleic acid. Each of the first target nucleic acid, the second target nucleic acid, and the reference nucleic acid has an expected concentration in the test sample that is within a dynamic range of the real-time quantitative isothermal amplification as verified over a cohort population of interest.

At 504, an aliquot of the test sample is added to at least one reaction vessel containing a master mix for isothermal amplification of the first target nucleic acid, the second target nucleic acid, and the reference nucleic acid.

At 506, at least one reaction of isothermal amplification is started in the at least one reaction vessel.

At 508, a first time-to-threshold value for the first target nucleic acid is determined in the at least one reaction.

At 510, a second time-to-threshold value for the second target nucleic acid is determined in the at least one reaction.

At 512, a reference time-to-threshold value for the reference nucleic acid is determined in the at least one reaction.

At 514, a first relative abundance value of the first target nucleic acid relative to the reference nucleic acid in the test sample is estimated based at least on the first time-to-threshold value and the reference time-to-threshold value.

At 516, a second relative abundance value of the second target nucleic acid relative to the reference nucleic acid in the test sample is estimated based at least on the second time-to-threshold value and the reference time-to-threshold value.

At 518, the diagnostic score of the test sample is estimated based on the first time-to-threshold value for the first target nucleic acid and the second time-to-threshold value for the second target nucleic acid.

In some embodiments, the method 500 further includes, before obtaining the test sample, performing real-time quantitative isothermal amplification across the cohort population of interest to establish a clinically relevant threshold diagnostic score; and after estimating the diagnostic score of the test sample, making a clinical diagnosis of a medical condition by comparing the diagnostic score of the test sample to the threshold diagnostic score.

In some embodiments, the isothermal amplification is loop-mediated isothermal amplification (LAMP).

In some embodiments, the diagnostic score relates to a difference between the first time-to-threshold value for the first target nucleic acid and the second time-to-threshold value for the second target nucleic acid.

In some embodiments, the test sample contains a plurality of first target nucleic acids and a plurality of second target nucleic acids. The diagnostic score relates to a difference between a first statistical value based on the time-to-threshold values of the plurality of first target nucleic acids and a second statistical value based on the time-to-threshold values of the plurality of second target nucleic acids.

It should be appreciated that the specific steps illustrated in FIG. 5 provide a particular method for estimating a diagnostic score by performing real-time quantitative isothermal amplification according to some embodiments of the present invention. Other sequences of steps may also be performed according to alternative embodiments. For example, alternative embodiments of the present invention may perform the steps outlined above in a different order. Moreover, the individual steps illustrated in FIG. 5 may include multiple stab-steps that may be performed in various sequences as appropriate to the individual step. Furthermore, additional steps may be added or removed depending on the particular applications. One of ordinary skill in the art would recognize many variations, modifications, and alternatives.

### L. Method of Estimating a Diagnostic Score Using Multi-Volume Real-Time Quantitative Isothermal Amplification Assays

According to some embodiments, a method of estimating a diagnostic score using multiple biomarkers may utilize a multi-volume real-time quantitative isothermal amplification approach as discussed above. For example, in HostDx-Fever qLAMP assays involving seven identified target genes IFI27, JUP, LAX1, TNIP1, GPAA1, CTSB, some of the seven target genes may be expected to be more abundant than some other target genes in a test sample. It may be advantageous to perform real-time quantitative isothermal amplification assays for those genes that are expected to be more abundant in larger-volume reaction vessels, and for those genes that are expected to be less abundant in smaller-volume reaction vessels. In this way, given a limited amount of test sample, quantitative isothermal amplification assays may be performed successfully for both genes with higher expected abundance and genes with lower expected abundance.

In some embodiments, the expected relative abundance for various genes may be established by a population test prior to well assignment. In the population test, real-time quantitative isothermal amplification assays are performed for the various genes in a clinically representative population large enough to establish statistical significance. For example, the population test may use a student's t-test, a Welch's t-test, a Mann-Whitney U test, or an analysis of variance (ANOVA), an F-test, or the like. The statistical significance may be set at, for example, p<0.05.

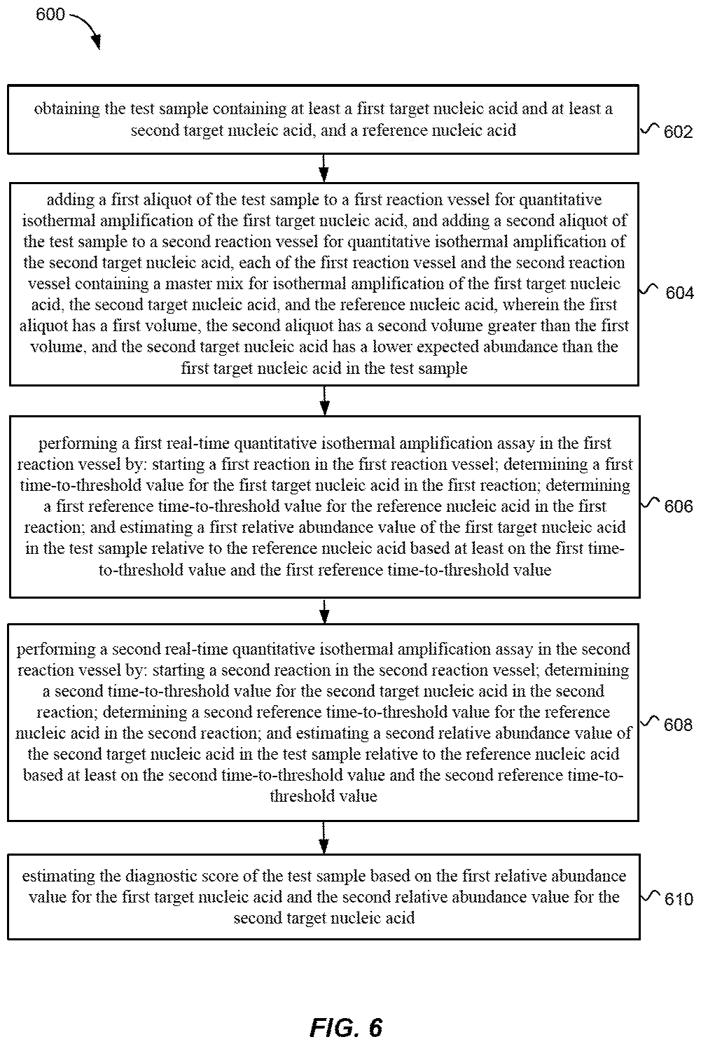

FIG. 6 shows a simplified flowchart illustrating a method of estimating a diagnostic score by performing real-time quantitative isothermal amplification on a test sample according to some embodiments.

At 602, the test sample is obtained. The test sample contains at least a first target nucleic acid and at least a second target nucleic acid, and a reference nucleic acid. Each of the first target nucleic acid, the second target nucleic acid, and the reference nucleic acid includes a mammalian host nucleic acid.

At 604, a first aliquot of the test sample is added to a first reaction vessel for quantitative isothermal amplification of the first target nucleic acid, and a second aliquot of the test sample is added to a second reaction vessel for quantitative isothermal amplification of the second target nucleic acid. Each of the first reaction vessel and the second reaction vessel contains a master mix for isothermal amplification of the first target nucleic acid, the second target nucleic acid, and the reference nucleic acid. The second target nucleic acid has a lower expected abundance than the first target nucleic acid in the test sample. The first aliquot has a first volume. The second aliquot has a second volume greater than the first volume.

At 606, a first real-time quantitative isothermal amplification assay is performed in the first reaction vessel by: starting a first reaction in the first reaction vessel; determining a first time-to-threshold value for the first target nucleic acid in the first reaction; determining a first reference time-to-threshold value for the reference nucleic acid in the first reaction; and estimating a first relative abundance value of the first target nucleic acid in the test sample relative to the reference nucleic acid based at least on the first time-to-threshold value and the first reference time-to-threshold value.

At 608, a second real-time quantitative isothermal amplification assay is performed in the second reaction vessel by: starting a second reaction in the second reaction vessel; determining a second time-to-threshold value for the second target nucleic acid in the second reaction; determining a second reference time-to-threshold value for the reference nucleic acid in the second reaction; and estimating a second relative abundance value of the second target nucleic acid in the test sample relative to the reference nucleic acid based at least on the second time-to-threshold value and the second reference time-to-threshold value.

At 610, the diagnostic score of the test sample is estimated based on the first relative abundance value for the first target nucleic acid and the second relative abundance value for the second target nucleic acid.

In some embodiments, method 600 further includes, before adding the first aliquot of the test sample to the first reaction vessel and adding the second aliquot of the test sample to the second reaction vessel, establishing that the second target nucleic acid has the lower expected abundance than the first target nucleic acid by performing test real-time quantitative isothermal amplification assays for the first target nucleic acid and the second target nucleic acid in a clinically representative population.

In some embodiments, the diagnostic score relates to a difference between the first relative abundance value and the second relative abundance value.

In some embodiments, the test sample contains a plurality of first target nucleic acids and a plurality of second target nucleic acids, and the diagnostic score relates to a difference between a first statistical value based on the relative abundance values of the plurality of first target nucleic acids and a second statistical value based on the relative abundance values of the plurality of second target nucleic acids. In some embodiments, the first statistical value includes a geometric mean of the relative abundance values of the plurality of first target nucleic acids, and the second statistical value includes a geometric mean of the relative abundance values of the plurality of second target nucleic acids. In some embodiments, the diagnostic score is used to diagnose whether a patient has a bacterial infection or a viral infection. In some embodiments, the plurality of first target nucleic acids includes genes that are higher in viral infections, and the plurality of second target nucleic acids includes genes that are higher in bacterial infectious. For example, the plurality of first target nucleic acids may include IFI27, JUP, and LAX1; and the plurality of second target nucleic acids may include HK3, TNIP1, GPAA1, and CTSB.

In some embodiments, the test sample contains a plurality of first target nucleic acids and a plurality of second target nucleic acids. The diagnostic score is estimated based on the relative abundance values of the plurality of first target nucleic acids and the relative abundance values of the plurality of second target nucleic acids using a regression model, a tree-based machine-learning model, a support vector machine model, or an artificial neural network (ANN) model.

In some embodiments, each of the first real-time quantitative isothermal amplification assay and the second real-time quantitative isothermal amplification assay is a real-time quantitative loop-mediated isothermal amplification (LAMP) assay.

In some embodiments, each of the first reaction in the first reaction vessel and the second reaction in the second reaction vessel is started using a hot start mechanism.

It should be appreciated that the specific steps illustrated in FIG. 6 provide a particular method for estimating a diagnostic score by performing real-time quantitative isothermal amplification according to some embodiments of the present invention. Other sequences of steps may also be performed according to alternative embodiments. For example, alternative embodiments of the present invention may perform the steps outlined above in a different order. Moreover, the individual steps illustrated in FIG. 6 may include multiple sub-steps that may be performed in various sequences as appropriate to the individual step. Furthermore additional steps may be added or removed depending on the particular applications. One Of ordinary skill in the art would recognize many variations, modifications, and alternatives.

### M. Platforms/Devices

In some aspects, the disclosure provides platforms or devices for obtaining a relative abundance value for a target nucleic acid in a test sample. Any suitable device or platform for performing the methods may be used. In some embodiments, the device is useful for relatively quantifying host mammalian nucleic acids in a test sample, where the host mammalian nucleic acids are reflective of the host having an acute infection, such as a bacterial or viral infection. In some embodiments, the acute infection has induced sepsis.

In some embodiments, the device is a microfluidic device. In some embodiments, the microfluidic device allows for amplification of a target nucleic acid and a reference nucleic acid in the same reaction vessel. In another embodiment, the microfluidic device allows for amplification of a target nucleic acid and a reference nucleic acid in distinct reaction vessels (e.g., separate wells).

Any device that is capable of heating a sample to a constant temperature and monitoring fluorescence, turbidity, luminescence, absorbance (color), or magnetic/electromagnetic current can be used for isothermal amplification. In some embodiments, the device is TaqMan Gene Expression Assays from ThermoFisher Scientific.

In some embodiments, the device for performing the method includes a fluorescent label detection system such as, but not limited to, Applied Biosystem QuantStudio Real-Time PCR System (under isothermal conditions).

As will be apparent to one of ordinary skill in the art, the platform or device used to perform the invention can include any useful dimensions (e.g., length, width, and depth). In some embodiments, the device is a bench-top size device that utilizes one or reaction vessels containing the target nucleic acid and the reference nucleic acid. In some embodiments, the reaction vessels are housed in a single unit, such as a 96-well plate. In some embodiments, the reaction vessels (or multiple housing units) can be stored, tested, and/or analyzed in the device sequentially or simultaneously

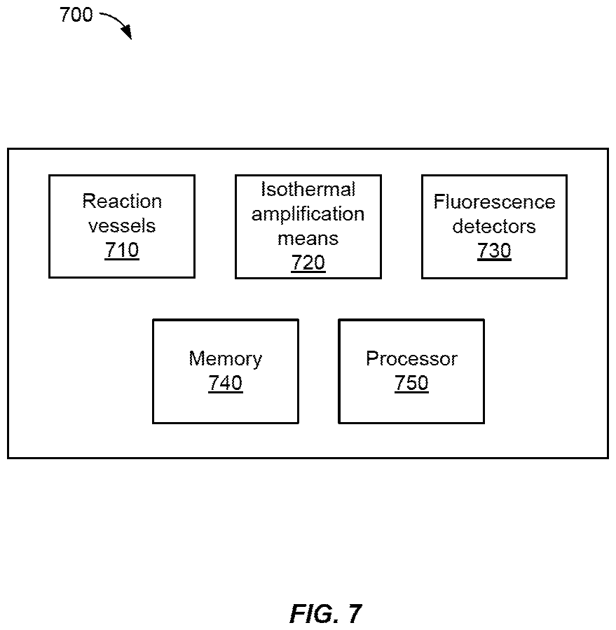

FIG. 7 shows a schematic block diagram of an apparatus 700 for estimating a relative abundance value using a real-time quantitative isothermal amplification assay according to some embodiments.

The apparatus 700 includes one Or more reaction vessels 710. Each reaction vessel 710 is configured to hold an aliquot of a test sample containing at least one target nucleic acid and a reference nucleic acid. Each reaction vessel 710 is further configured to hold a master mix for isothermal amplification of the at least one target nucleic acid and the reference nucleic acid. The master mix also includes fluorescence labels for detecting the at least one target nucleic acid and the reference nucleic acid.

In some embodiments, the one or more reaction vessels 710 include at least a first reaction vessel and a second reaction vessel. The first reaction vessel may be configured for holding a first aliquot of the test sample and a first portion of the master mix for isothermal amplification of a first target nucleic acid. The second reaction vessel may be configured for holding a second aliquot of the test sample and a second portion of the master mix for isothermal amplification of a second target nucleic acid. In some embodiments, the one or more reaction vessels 710 include a third reaction vessel configured for holding a third aliquot of the test sample and a third portion of the master mix for isothermal amplification of the reference nucleic acid.

The apparatus 700 further includes isothermal amplification means 720 for starting isothermal amplification reactions in the one or more reaction vessels 710. For example, the isothermal amplification means 720 may include heating elements and temperature controls to heat the reaction vessel 710 and its content to a temperature necessary for starting isothermal amplification of the at least one target nucleic acid and the reference nucleic acid. The isothermal amplification reaction may produce fluorescence associated with the at least one target nucleic acid and fluorescence associated with the reference nucleic acid.

The apparatus 700 further includes one or more fluorescence detectors 730 optically coupled to the one or more reaction vessels 710. Each fluorescence detector 730 is configured to detect the fluorescence associated with a respective target nucleic acid or the fluorescence associated with the reference nucleic acid in real time during the isothermal amplification reaction. Thus, an intensity of the fluorescence associated with the respective target nucleic acid may be measured as a function of time, and an intensity of the fluorescence associated with the reference nucleic acid may be measured as a function of time. In some embodiments, each fluorescence detector is configured to detect fluorescence associated with the respective target nucleic acid at regular intervals during the isothermal amplification reaction. For example, a regular interval of fluorescence detection may occur once every minute, once every 30 seconds, once every 20 seconds, once every 10 seconds, once every 5 seconds, or once every second, during the isothermal amplification reaction.

The apparatus 700 further includes a computer memory 740. In some embodiments, the computer memory is configured to store one or more standard curves. For example, a first standard curve may provide a first function relating starting number of copies of a first target nucleic acid to time-to-threshold. A second standard curve may provide a second function relating starting number of copies of a second target nucleic acid to time-to-threshold. A third standard curve may provide a third function relating starting number of copies of a reference nucleic acid to time-to-threshold. The first standard curve, the second standard curve, and the third standard curve may be obtained from previous isothermal amplification reactions of a calibrator sample, and are stored in the memory 740 for use in subsequent isothermal amplification reactions of test samples. In some embodiments, the computer memory 740 is configured to store one or more threshold fluorescence intensity values.

The apparatus 700 further includes a computer processor 750 coupled to the fluorescence detector 730 and the memory 740. The memory 740 may further store instructions to be executed by the computer processor 750.

In some embodiments, the processor 750 is configured to determine a first time-to-threshold value for a first target nucleic acid based on the intensity of the fluorescence associated with the first target nucleic acid as a function time and the stored first threshold fluorescence intensity value, and estimate a starting number of copies of the first target nucleic acid in the test sample based on the first time-to-threshold value using the first function provided by the first standard curve. The processor 750 is further configured to determine a second time-to-threshold value for a second target nucleic acid based on the intensity of the fluorescence associated with the second target nucleic acid as a function time and the stored second threshold fluorescence intensity value, and estimate a starting number of copies of the second target nucleic acid in the test sample based on the second time-to-threshold value using the first function provided by the second standard curve. The processor 750 is further configured to determine a third time-to-threshold value for the reference nucleic acid based on the intensity of the fluorescence associated with the reference nucleic acid as a function time and the stored third threshold fluorescence intensity value, and estimate a starting number of copies of the reference nucleic acid in the test sample based on the third time-to-threshold value using the third function provided by the third standard curve. The processor 750 is further configured to estimate a relative abundance value of the first target nucleic acid in the test sample relative to the reference nucleic acid based on the starting number of copies of the first target nucleic acid and the starting number of copies of the reference nucleic acid, and estimate a relative abundance value of the second target nucleic acid in the test sample relative to the reference nucleic acid based on the starting number of copies of the second target nucleic acid and the starting number of copies of the reference nucleic acid. The processor 750 may be configured to estimate a diagnostic score of the test sample based on the first relative abundance value for the first target nucleic acid and the second relative abundance value for the second target nucleic acid.

### N. Kits

In some aspects, the disclosure provides kits for calculating a relative abundance value of a target nucleic acid in a test sample. In some embodiments, the kits comprise at least one reaction vessel, and one or more components for performing real-time quantitative isothermal amplification (e.g., a RNA polymerase, reverse transcriptase and/or DNA polymerase). In some embodiments, the kit can include two or more reaction vessels (e.g., a first reaction vessel and a second reaction vessel). In some embodiments, the kit comprises a master mix for isothermal amplification. In some embodiments, the kit comprises a master mix for real-time quantitative isothermal amplification. The kit may be accompanied by instructions for interpretation of results from the real-time quantitative isothermal amplification assay. Instructions (e.g., written, CD-ROM, etc.,) for carrying out the real-time quantitative isothermal amplification assay may also be included in the kit.

### EXAMPLES

The following examples are offered to illustrate, but not to limit the claimed invention.

### Example 1: Isothermal Amplification Assay Setup

Isothermal assay pruners comprise a forward inner primer (FIP) and forward outer primer (F3) and corresponding backward inner primer (BIP) and backward outer primer (B3), along with forward and backward rate enhancing primers (FR, BR). Assays are carried out using WarmStart (also referred in as hot start) LAMP 2X Master Mix (NEB, CAT# E1700S) with the Manufacturer's protocol adjusted for 20 μL total reaction volume and with the optional fluorescent dye added to a final concentration of 1×. Primers are added to final concentrations of 1.6 μM FIP/BIP, 0.2 μM F3/B3, and 0.4 μM FR/BR. Assays are supplemented with 1 mM dUTP (ThermoFisher CAT# R0133) to improve assay fidelity. Template is added in a standard 1 μL volume containing an empirically optimized mass of sample material. Finally, water is added to bring the final volume to 20 μL per reaction. Assays are distributed in 96-well plates (ThermoFisher CAT# 4346906) for quantitative amplification using a real-time PCR instrument.

Quantitative isothermal amplification is performed on a QuantStudio 6 Flex Real-Time PCR System (Thermo Fisher) using the FAM/SYBR Green channel to monitor dye fluorescence. Cycling conditions include a 5 min hold step at 25° C. followed by a 60 min hold step at 65° C. during which fluorescence is monitored at 20 second intervals. Use of the NEB kit containing a warm start polymerase ensures that isothermal reactions are initiated only when the solution temperature reaches 45° C. This allows comparison of assays across plates/runs by ensuring that an equivalent time to threshold (Tt) value determined in different runs represents an equal time lapse from reaction initiation across those runs. This also allows assays to be calibrated to a previously determined standard curve such that assays of varying efficiency can be compared for relative target quantitation.

### Example 2: Obtaining Standard Curves And Calculating Relative Abundance

To calibrate isothermal amplification assays, double-stranded DNA template corresponding to mRNA sequences of interest is synthesized by a commercial vendor (e.g., Integrated DNA Technologies) to be used as quantitated control samples. Lyophilized synthetic templates are resuspended in a TE buffer {20 mM Tris pH 8.0, 0.5 mM EDTA} to a final concentration of approximately 1×10⁹ copies/μL. The precise concentration of each template is determined using a Qubit Fluorimeter and associated Qubit dsDNA HS Assay Kit (e.g., ThermoFisher Scientific, CAT# Q32851) according to the manufacturer's protocol. This value is used in subsequent regression analysis. To produce samples for standard curve analysis, each template is serially diluted in a TE buffer at 10-fold concentration intervals yielding samples covering a range from approximately 10⁹ copies/μL to approximately 10² copies/μL. A time to threshold value (Tt) is determined for each sample in triplicate using 1 μL input as a template for the corresponding isothermal amplification assay.

### Example 3: Selection of Threshold Intensity

Threshold values are a function of the fluorescent dye or probe being used and the instrument on which the fluorescence is being detected. As a general principal, threshold values are determined by identifying a point in the linear phase of amplification that is both significantly above background signal and maintains the lowest standard deviation in resultant time to threshold (Tt) across multiple experimental replicates. The same threshold value must be used for a given target both when determining the standard curve and when measuring target abundance in a sample of interest. While it is not necessary to use the same threshold for different targets when a unique standard curve is determined for each target, it is important to maintain the same threshold value for all targets in a relative comparison that does not proceed through standard curve calibration.

### Example 4: Relative Target Nucleic Acid Quantitation by Isothermal Amplification and Comparison to a Gold Standard mRNA Quantitation Assay

To evaluate the performance of relative mRNA quantitation from patient blood samples using isothermal amplification assays as described herein, relative abundance values of a target nucleic acid (referred to herein as ‘biomarker IFI27’) relative to a reference nucleic acid (referred to herein as ‘housekeeping gene YWHAB’), were determined and compared to measurements obtained using a gold standard mRNA quantitation assay, conducted on a NanoString nCounter (NanoString Technologies Inc., Seattle). NanoString is highly accurate and is a useful tool for measuring the expression levels of multiple genes at once; however, it is also likely too slow for clinical application (4-6 hours per assay).

### Sample Selection and RNA Extraction

To recapitulate the range of target nucleic acid abundance that would be expected in a clinical setting, samples were selected from patients presenting with viral infection (6) or bacterial sepsis (8), in addition to a set of healthy controls (6) (see, Table 1). Samples were collected in PAXgene Blood RNA Tubes (PreAnalytiX, GmbH) and treated per the manufacturer's protocol prior to storage at −80° C.

At the time of analysis, samples were thawed for 2 hours at room temperature, then inverted greater than 10 times to homogenize the contents of the vacutainer. Final RNA purification was performed on aliquots of each sample using a Qiacube (Qiagen, Maryland), and purified total RNA was quantitated using a Qubit fluorimeter (ThermoFisher Scientific, Waltham) (See, Table 1).

### Results—Relative Abundance Evaluations

To evaluate the relative abundance of the target nucleic acid relative to the reference nucleic acid, each target nucleic acid or reference nucleic acid was amplified in single-plea from 50 ng of total RNA using the one-step reverse transcription-isothermal amplification assay described above.

Time to threshold (Tt) values were determined for each target nucleic acid and each reference nucleic acid in each sample, then converted to Loge (Template Copy Number) using a previously determined standard curve. The relative abundance of IFI27 to YWHAB was then calculated by taking the ratio of the two measurements (see, Table 1).

The same RNA samples were also analyzed on a NanoString nCounter, which provides a direct quantitation of host mRNA transcripts present in a sample without amplification. Transcript abundance values for IFI27 and YWHAB obtained using the NanoString nCounter were log-transformed, and the ratio of these values was determined and compared to measurements obtained using the isothermal amplification assay described above.

To quantify performance of the isothermal amplification assay relative to the gold standard, relative abundance values determined using the isothermal amplification assay were plotted as a function of values determined using the NanoString nCounter, and the Pearson correlation coefficient between these measurements across all samples was calculated (see, FIG. 7).

TABLE 1
Clinical sample diagnosis, target nucleic acid and reference nucleic acid abundance

| | | Isothermal Assay | | | | | | Nano String ttCountter | | |
|---|---|---|---|---|---|---|---|---|---|---|
| | | RNA | | Calculated | YWHAB | Calculated | Relative | IFI27 | YWHAB | Relative |
| | | recovery | IFI27T [symbol] | IFI27 | T [symbol] | YWHAB | IFI27/ | Abundance | Abundance | IFI27/ |
| Sample | Diagnosis | (ng) | (min) | Abundance | (min) | Abundance | YWHAB | (Counts) | (Counts) | YWHAB |
| 20125 | Viral | 1896 | 11.40 | 6850649 | 12.60 | 179465 | 5.25 | 84811 | 3748 | 4.50 |
| 20126 | Viral | 1136 | 11.43 | 6514232 | 12.06 | 419991 | 3.96 | 77389 | 3956 | 4.29 |
| 20128 | Viml | 896 | 13.79 | 201436 | 12.08 | 402774 | −1.00 | 1934 | 5376 | −1.48 |
| 20129 | Viral | 1000 | 12.02 | 2759348 | 12.19 | 838540 | 3.03 | 33614 | 5402 | 2.64 |
| 20130 | Viral | 1420 | 11.93 | 3109737 | 12.16 | 354499 | 3.13 | 20675 | 2074 | 3.32 |
| 20131 | Viral | 864 | 11.62 | 4926631 | 12.16 | 354098 | 3.80 | 50057 | 1944 | 4.69 |
| P3342 | Bacterial | 1460 | 14.34 | 89789 | 12.14 | 367499 | −2.03 | 1017 | 7301 | −2.84 |
| | Sepsis | | | | | | | | | |
| P3345 | Bacterial | 368 | 16.59 | 3236 | 12.67 | 160538 | −5.63 | 189 | 7211 | −5.26 |
| | Sepsis | | | | | | | | | |
| P3405 | Bacterial | 3360 | 14.51 | 69312 | 12.11 | 387937 | −2.48 | 1017 | 7950 | −2.97 |
| | Sepsis | | | | | | | | | |
| P3502 | Bacterial | 1888 | 15.25 | 23247 | 12.22 | 322669 | −3.79 | 789 | 11982 | −3.92 |
| | Sepsis | | | | | | | | | |
| P3505 | Bacterial | 2480 | 12.39 | 1594857 | 12.80 | 131241 | 3.60 | 44020 | 3279 | 3.75 |
| | Sepsis | | | | | | | | | |
| P3512 | Bacterial | 3840 | 14.62 | 59347 | 12.02 | 447708 | −2.52 | 1088 | 7949 | −2.87 |
| | Sepsis | | | | | | | | | |
| P3602 | Bacterial | 2800 | 17.99 | 411 | 12.24 | 314923 | −9.58 | 66 | 11183 | −7.65 |
| | Sepsis | | | | | | | | | |
| P3952 | Bacterial | 2880 | 16.80 | 2362 | 11.82 | 607929 | −8.01 | 96 | 8142 | −6.40 |
| | Sepsis | | | | | | | | | |
| 10 | Healthy | 2920 | 13.88 | 176617 | 11.96 | 488427 | −1.47 | 5542 | 11298 | −1.03 |
| 11 | Healthy | 2000 | 14.63 | 58466 | 12.34 | 268557 | −2.20 | 1913 | 5157 | −1.43 |
| 12 | Healthy | 1536 | 16.67 | 2883 | 12.24 | 315862 | −6.78 | 235 | 5938 | −4.66 |
| 13 | Healthy | 1600 | 16.47 | 3861 | 12.13 | 371597 | −6.59 | 242 | 5870 | −4.60 |
| 14 | Healthy | 1684 | 15.56 | 14750 | 12.46 | 223964 | −3.92 | 487 | 6895 | −3.82 |
| 16 | Healthy | 4000 | 15.35 | 20295 | 12.15 | 363015 | −4.16 | 627 | 4275 | −2.77 |

[symbol]

indicates data missing or illegible when filed

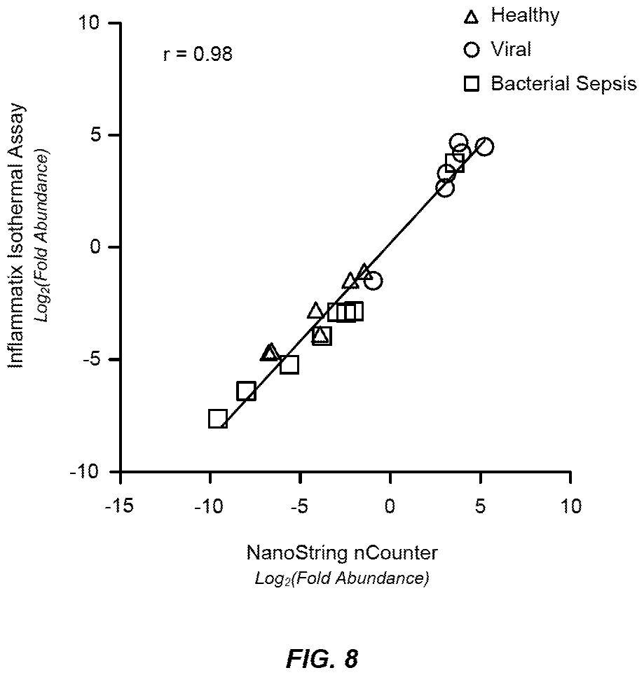

FIG. 8 illustrates the correlation plot with Pearson coefficient relating target nucleic acid (IFI27) abundance relative to reference nucleic acid (YWHAB) abundance determined by the isothermal amplification assay and the gold standard NanoString nCounter. Values are plotted as Log₂(Fold Abundance) for both assays and coded according to diagnosis: circles represent viral infection; squares represent bacterial sepsis; and triangles represent healthy controls. The line represents a curve fit by standard linear regression analysis for ease of interpretation.

The isothermal amplification assay demonstrated exceptional correlation with the gold standard assay (r=0.97), indicating excellent agreement in relative abundance measurements. Importantly, groups representing different diagnoses (e.g., bacterial sepsis, viral infection or healthy sample) clustered for both assays, indicating clinical utility is maintained when using the isothermal amplification assay.

### Example 5: HostDx-Fever qLAMP Assays and Comparison to a Gold Standard mRNA Quantitation Assay

A set of seven genes was previously identified as biomarkers useful for classifying viral infections or bacterial infections. The seven genes include IFI27, ATP, LAX1, which are higher in viral infections, and HK3, TNIP1, GPAA1, CTSB, which are higher in bacterial infections. (See U.S. Patent Application Publication 2019/0144943.) An integrated diagnostic score (referred to as the fever score or Host-Dx-Fever score) based on the levels of the seven biomarkers may be estimated using a difference of geometrical means (DGM), as expressed according to the formula:

$$\sqrt[3]{IFI 27 * JUP * LAX 1} - \sqrt[4]{HK 3 * TNIP 1 * GPAA 1 * CTSB} . \qquad ( 8 )$$

In an experiment, HostDx-Fever qLAMP assays were validated for each of the seven target genes across an analytical validation cohort. Total RNA was extracted and LAMP assays were performed in triplicate on a QuantStudio6 qPCR machine. The analytical gold standard was the NanoString nCounter absolute mRNA counts. For both technologies, the HostDx-Fever score was calculated as the DGM of the mRNA assays as expressed in Equation (8).

### Relative Quantification Using Isothermal Amplification Without Standard Curves

In the experiment, the same bacterial and viral clinical samples were tested with two sets of assays. Each set of assays targeted the seven previously identified biomarkers that, when combined using the DGM approach, creates a single diagnostic score that can separate bacterial from viral infections. The first set of assays used the NanoString nCounter digital mRNA platform, where housekeeper-normalized counts are fed into the DGM equation (e.g., Equation (8)).

The second set of assays was a set of isothermal qLAMP assays targeting the same mRNAs. The time-to-threshold value Tt for each assay was fed into the DGM equation (e.g., Equation (8)). Note that the time-to-threshold values are directly fed into the DGM equation without being converted into absolute copy numbers using a standard curve.

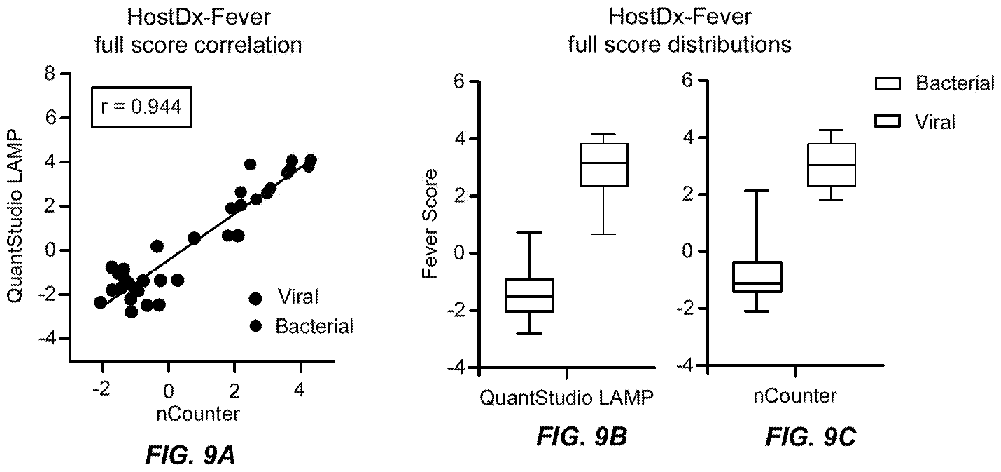

FIG. 9A shows the correlation plot with Pearson coefficient relating the diagnostic scores determined by the isothermal amplification assays and by the gold standard NanoString nCounter. The black circles represent viral infection; and the gray circles represent bacterial infection. The straight line represents a curve fit by standard linear regression analysis for ease of interpretation, the isothermal amplification assays demonstrated exceptional correlation with the gold standard assay (r=0.944). This shows that the qLAMP technology even without using standard curves can yield highly accurate diagnostic results in relevant clinical samples against an analytical gold standard.

FIG. 9B shows the HostDx-Fever score distribution based on qLAMP assays. FIG. 9C shows the HostDx-Fever score distribution based on NanoString nCounter. Note that groups representing different diagnoses (e.g., bacterial infection or viral infection) clustered for both types of assays. This shows that HostDx-Fever run on either technology can accurately discriminate between infection classes, and that the HostDx-Fever biomarkers are relatively insensitive to underlying quantitative technology. However, the optimal clinical cutoff may be different for the two technologies, as the technical platforms can give rise to different absolute numbers even while maintaining similar relative quantification.

### Example 6: Multi-Volume Isothermal qLAMP Assays

An experiment was conducted to test whether interrogating larger volumes at a given template concentration may improve success rate of qLAMP assays at the lower end of the linear dynamic range. The hypotheses are: (i) qLAMP, similar to qPCR, measures the time required to accumulate an arbitrary but fixed threshold number of copies of a target gene in a reaction vessel; (ii) under saturating binding conditions, the time required to reach the threshold number depends on (a) the number of copies present when the reaction is initiated, and (b) the rate of increase per unit time (which is referred to as reaction efficiency or amplification efficiency).

In the experiment, identical concentration of template is maintained across two different volumes: 10 μL and 50 μL. An isothermal amplification is considered successful if the measured time-to-threshold value Tt falls under a predefined cutoff value. The cutoff value may be defined as the y-intercept of a linear regression fit to a standard curve titration, as illustrated below, or by other means.

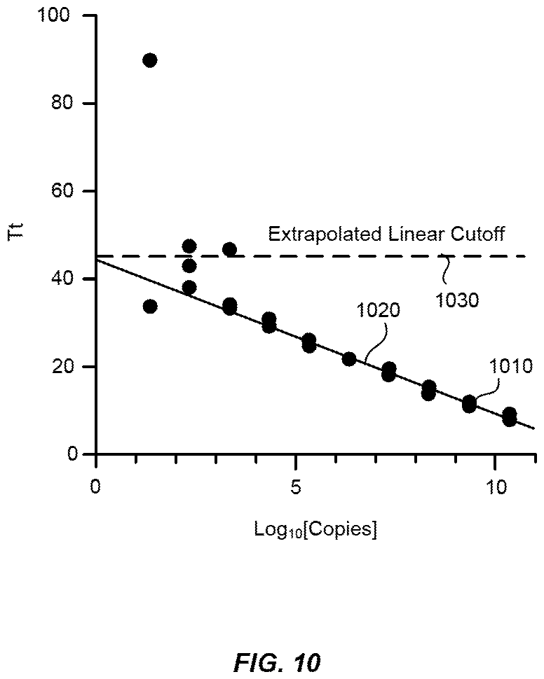

FIG. 10 illustrates an example in which the cutoff value is defined as the y-intercept of a linear regression fit to a standard curve titration. The circles 1010 are the measured time-to-threshold values for various input concentrations. The straight line 1020 represents the standard curve obtained by the linear regression fit. The horizontal dashed line 1030 indicates the y-intercept (e.g., the value of “b” in the standard curve as expressed in Equation (1) or Equation (2)). Thus, in some embodiments, if a measured time-to-threshold value Tt falls under the horizontal dashed line 930, the isothermal amplification may be considered as successful.

In the experiment, on a 96-well plate, each of 48 wells is filled with a respective first aliquot of 10 μL of the test sample, and each of the other 48 wells is filled with a respective second aliquot of 50 μL of the test sample. The first aliquot and the second aliquot have the same template concentration.

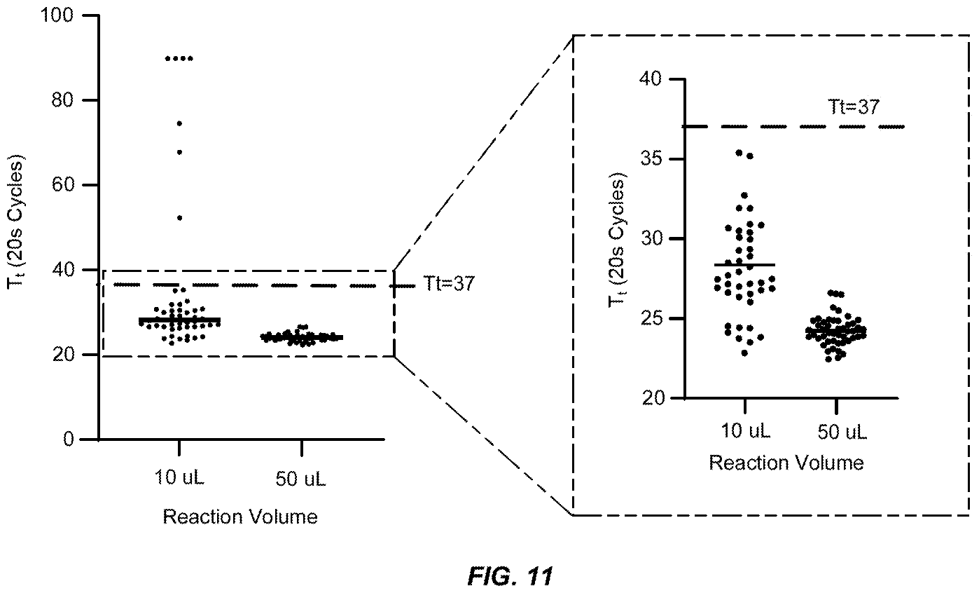

FIG. 11 shows the experimental results of the qLAMP assays. The black circles represent the measured time-to-threshold values Tt for the 20-μL reaction volumes; and the gray circles represent the measured time-to-threshold values Tt for the 50-μL reaction volumes. The inset shows an enlarged version for the time-to-threshold range between 20 and 40 time cycles (20 seconds each). The dashed horizontal line indicates the predefined cutoff Tt value, as discussed above. As illustrated, the higher-volume reactions display earlier time-to-threshold values and a higher success rate.

The experiment demonstrates that, for a fixed number of template molecules distributed in a given volume, if the fixed number is near the lower end of the linear dynamic range, interrogating a larger fraction of the volume will have a higher probability of generating an accurate (linear) measurement. Thus, using larger well volumes for assaying low abundance biomarkers may improve the linear dynamic range, extending the number of samples that can be successfully nm with the assay.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for estimating a diagnostic score of a test sample using real-time quantitative isothermal amplification, the method comprising:
obtaining the test sample containing at least a first target nucleic acid and at least a second target nucleic acid, and a reference nucleic acid, wherein each of the first target nucleic acid, the second target nucleic acid, and the reference nucleic acid comprises a mammalian host nucleic acid;
adding a first aliquot of the test sample to a first reaction vessel for quantitative isothermal amplification of the first target nucleic acid, and adding a second aliquot of the test sample to a second reaction vessel for quantitative isothermal amplification of the second target nucleic acid, each of the first reaction vessel and the second reaction vessel containing a master mix for isothermal amplification of the first target nucleic acid, the second target nucleic acid, and the reference nucleic acid, wherein the second target nucleic acid has a lower expected abundance than the first target nucleic acid in the test sample, the first aliquot has a lint volume, and the second aliquot has a second volume greater than the first volume;
performing a first real-time quantitative isothermal amplification assay in the first reaction vessel by:
starting a first reaction in the first reaction vessel;
determining a first time-to-threshold value for the first target nucleic acid in the first reaction;
determining a first reference time-to-threshold value for the reference nucleic acid in the first reaction; and
estimating a first relative abundance value of the first target nucleic acid in the test sample relative to the reference nucleic acid based at least on the first time-to-threshold value and the first reference time-to-threshold value;
performing a second real-time quantitative isothermal amplification assay in the second reaction vessel by:
starting a second reaction in the second reaction vessel;
determining a second time-to-threshold value for the second target nucleic acid in the second reaction;
determining a second reference time-to-threshold value for the reference nucleic acid in the second reaction; and
estimating a second relative abundance value of the second target nucleic acid in the test sample relative to the reference nucleic acid based at least on the second time-to-threshold value and the second reference time-to-threshold value; and
estimating the diagnostic score of the test sample based on the first relative abundance value for the first target nucleic acid and the second relative abundance value for the second target nucleic acid.

2. The method of claim 1, further comprising, prior to adding the first aliquot of the test sample to the first reaction vessel and adding the second aliquot of the test sample to the second reaction vessel, establishing that the second target nucleic acid has the lower expected abundance than the first target nucleic acid by performing test real-time quantitative isothermal amplification assays for the first target nucleic acid and the second target nucleic acid in a clinically representative population.

3. The method of claim 1, wherein the diagnostic score relates to a difference between the first relative abundance value and the second relative abundance value.

4. The method of claim 1, wherein the test sample contains a plurality of first target nucleic acids and a plurality of second target nucleic acids, and the diagnostic score relates to a difference between a first statistical value based on the relative abundance values of the plurality of first target nucleic acids and a second statistical value based on the relative abundance values of the plurality of second target nucleic acids.

5. The method of claim 4, Wherein the first statistical value comprises a geometric mean of the relative abundance values of the plurality of first target nucleic acids, and the second statistical value comprises a geometric mean of the relative abundance values of the plurality of second target nucleic acids.

6. The method of claim 4, wherein the first statistical value comprises an arithmetic mean or a sum of the relative abundance values of the plurality of first target nucleic acids, and the second statistical value comprises an arithmetic mean or a sum of the relative abundance values of the plurality of second target nucleic acids.

7. The method of claim 4, wherein the diagnostic score is used to diagnose whether a patient has a bacterial infection or a viral infection.

8. The method of claim 7, wherein the plurality of first target nucleic acids comprises genes that are higher in viral infections, and the plurality of second target nucleic acids comprises genes that are higher in bacterial infections.

9. The method of claim 8, wherein the plurality of first target nucleic acids comprises IFI27, JUP, and LAX1, and the plurality of second target nucleic acids comprises HK3, TNIP1, GPAA1, and CTSB.

10. The method of claim 1, wherein the test sample contains a plurality of first target nucleic acids and a plurality of second target nucleic acids, and the diagnostic score is estimated based on the relative abundance values of the plurality of first target nucleic acids and the relative abundance values of the plurality of second target nucleic acids using a regression model, a tree-based machine-learning model, a support vector machine model, or an artificial neural network (ANN) model.

11. The method of claim 1, wherein each of the first real-time quantitative isothermal amplification assay and the second real-time quantitative isothermal amplification assay is a real-time quantitative loop-mediated isothermal amplification (LAMP) assay.

12. The method of claim 1, wherein each of the first reaction in the first reaction vessel and the second reaction in the second reaction vessel is started using a hot start mechanism.

13. A method of estimating a diagnostic score using real-time quantitative isothermal amplification, the method comprising:
obtaining a test sample from a mammalian subject, the test sample containing at least a first target nucleic acid and at least a second target nucleic acid, and a reference nucleic acid, wherein each of the first target nucleic acid, the second target nucleic acid, and the reference nucleic acid has an expected concentration in the test sample that is within a dynamic range of the real-time quantitative isothermal amplification as verified over a cohort population of interest;
adding an aliquot of the test sample to at least one reaction vessel containing a master mix for isothermal amplification of the first target nucleic acid, the second target nucleic acid, and the reference nucleic acid;
starting at least one reaction of isothermal amplification in the at least one reaction vessel;
determining a first time-to-threshold value for the first target nucleic acid in the at least one reaction;
determining a second time-to-threshold value for the second target nucleic acid in the at least one reaction;
determining a reference time-to-threshold value for the reference nucleic acid in the at least one reaction;
estimating a first relative abundance value of the first target nucleic acid relative to the reference nucleic acid in the test sample based at least on the first time-to-threshold value and the reference time-to-threshold value;
estimating a second relative abundance value of the second target nucleic acid relative to the reference nucleic acid in the test sample based at least on the second time-to-threshold value and the reference time-to-threshold value; and
estimating the diagnostic score of the test sample based on the first time-to-threshold value for the first target nucleic acid and the second time-to-threshold value for the second target nucleic acid.

14. The method of claim 13, further comprising:
before obtaining the test sample, performing real-time quantitative isothermal amplification across the cohort population of interest to establish a clinically relevant threshold diagnostic score; and
after estimating the diagnostic score of the test sample, making a clinical diagnosis of a medical condition by comparing the diagnostic score of the test sample to the threshold diagnostic score.

15. The method of claim 13, wherein the isothermal amplification is loop-mediated isothermal amplification (LAMP).

16. The method of claim 13, wherein the diagnostic score relates to a difference between the first time-to-threshold value for the first target nucleic acid and the second time-to-threshold value for the second target nucleic acid.

17. The method of claim 13, wherein the test sample contains a plurality of first target nucleic acids and a plurality of second target nucleic acids, and the diagnostic score relates to a difference between a first statistical value based on the time-to-threshold values of the plurality of first target nucleic acids and a second statistical value based on the time-to-threshold values of the plurality of second target nucleic acids.

18. The method of claim 13, wherein the at least one reaction of isothermal amplification in the at least one reaction vessel is started using a hot start mechanism.

19. A method for estimating a diagnostic score by performing real-time quantitative isothermal amplification on a test sample, the method comprising:
obtaining a first standard curve comprising a first function relating starting number of copies of a first target nucleic acid to time-to-threshold, a second standard curve comprising a second function relating starting number of copies of a second target nucleic acid to time-to-threshold, and a reference standard curve comprising a reference function relating starting number of copies of a reference nucleic acid to time-to-threshold, wherein the first standard curve, the second standard curve, and the reference standard curve are generated prior to performing real-time quantitative isothermal amplification on the test sample;
obtaining the test sample from a mammalian subject, the test sample containing the first target nucleic acid, the second target nucleic acid, and the reference nucleic acid;
adding the test sample to at least one reaction vessel containing a master mix for isothermal amplification of the first target nucleic acid, the second target nucleic acid, and the reference nucleic acid;
starting at least one reaction of isothermal amplification in the at least one reaction vessel;
determining a first time-to-threshold value for the first target nucleic acid in the at least one reaction;
determining a second time-to-threshold value for the second target nucleic acid in the at least one reaction;
determining a reference time-to-threshold value for the reference nucleic acid in the at least one reaction;
estimating a first starting number of copies of the first target nucleic acid in the test sample based on the first time-to-threshold value using the first function of the first standard curve;
estimating a second starting number of copies of the second target nucleic acid in the test sample based on the second time-to-threshold value using the second function of the second standard curve;
estimating a reference starting number of copies of the reference nucleic acid in the test sample based on the reference time-to-threshold value using the reference function provided by the reference standard curve;
estimating a first relative abundance value of the first target nucleic acid in the test sample relative to the reference nucleic acid based on the first starting number of copies of the first target nucleic acid and the reference starting number of copies of the reference nucleic acid;
estimating a second relative abundance value of the second target nucleic acid in the test sample relative to the reference nucleic acid based on the second starting number of copies of the second target nucleic acid and the reference starting number of copies of the reference nucleic acid;
estimating the diagnostic score of the test sample based on the first relative abundance value of the first target nucleic acid and the second relative abundance value of the second target nucleic acid; and
making a clinical diagnosis of a medical condition by comparing the diagnostic score of the test sample a predetermined threshold diagnostic score.

20. The method of claim 19, wherein the isothermal amplification is loop-mediated isothermal amplification (LAMP).

21. The method of claim 19, wherein the at least one reaction of isothermal amplification in the at least one reaction vessel is started using a hot start mechanism.

22. The method of claim 19, wherein the diagnostic score relates to a difference between the first time-to-threshold value for the first target nucleic acid and the second time-to-threshold value for the second target nucleic acid.

23. An apparatus for estimating a diagnostic score of a test sample using real-time quantitative isothermal amplification, the apparatus comprising:
a first reaction vessel configured to hold a first aliquot of the test sample, and a second reaction vessel configured to hold a second aliquot of the test sample;
wherein the test sample contains a first target nucleic acid, a second target nucleic acid, and a reference nucleic acid, each of the first target nucleic acid, the second target nucleic acid, and the reference nucleic acid comprising a mammalian host nucleic acid:
wherein each of the first reaction vessel and the second reaction vessel contains a master mix for isothermal amplification of the first target nucleic acid, the second target nucleic acid, and the reference nucleic acid; and
wherein the first aliquot has a first volume, the second aliquot has a second volume greater than the first volume, and the second target nucleic acid has a lower expected abundance than the first target nucleic acid in the test sample;
a computer memory for storing a first threshold fluorescence intensity value and a second threshold fluorescence intensity value;
means for starting a first isothermal amplification reaction in the first reaction vessel amplifying the first target nucleic acid and the reference nucleic acid, the first isothermal amplification reaction producing first fluorescence associated with the first target nucleic acid and first reference fluorescence associated with the reference nucleic acid;
a first fluorescence detector and a first reference fluorescence detector optically coupled to the first reaction vessel, the first fluorescence detector configured to measure an intensity of the first fluorescence as a function of time and to measure a first reference intensity of the first reference fluorescence as a function of time;
means for starting a second isothermal amplification reaction in the second reaction vessel amplifying the second target nucleic acid and the reference nucleic acid, the second isothermal amplification reaction producing second fluorescence associated with the second target nucleic acid and second reference fluorescence associated with the reference nucleic acid;
a second fluorescence detector and a second reference fluorescence detector optically coupled to the second reaction vessel, the second fluorescence detector configured to measure an intensity of the second fluorescence as a function of time and to measure a second reference intensity of the second reference fluorescence as a function of time; and
a computer processor coupled to the first fluorescence detector, the second fluorescence detector, the first reference fluorescence detector, the second reference fluorescence detector, and the computer memory, the computer processor configured to:
determine a first time-to-threshold value for the first target nucleic acid based on the intensity of the first fluorescence as a function time and the fast threshold fluorescence intensity value;
determine a first reference time-to-threshold value for the reference nucleic acid based on the intensity of the first reference fluorescence as a function of time and the first threshold fluorescence intensity value;
estimate a first relative abundance value of the first target nucleic acid in the test sample relative to the reference nucleic acid based at least on the first time-to-threshold value and the first reference time-to-threshold value;
determine a second time-to-threshold value for the second target nucleic acid based on the intensity of the second fluorescence as a function of time and the second threshold fluorescence intensity value;
determine a second reference time-to-threshold value for the reference nucleic acid based on the intensity of the second reference fluorescence as a function of time and the second threshold fluorescence intensity value;
estimate a second relative abundance value of the second target nucleic acid in the test sample relative to the reference nucleic acid based at least on the second time-to-threshold value and the second reference time-to-threshold value; and
estimate the diagnostic score of the test sample based on the first relative abundance value for the first target nucleic acid and the second relative abundance value for the second target nucleic acid.

24. The apparatus of claim 23, wherein the master mix comprises a first fluorescent label for detecting the first target nucleic acid, a second fluorescent label for detecting the second target nucleic acid, and a third fluorescence label for detecting the reference nucleic acid.

25. The apparatus of claim 24, wherein each of the first fluorescence label, the second fluorescence label, and the third fluorescence label comprises a SYTO dye.

26. The apparatus of claim 23, wherein the first threshold fluorescence intensity value and the second threshold fluorescence intensity value are the same.

27. The apparatus of claim 23, wherein the first threshold fluorescence intensity value and the second threshold fluorescence intensity value are different from each other.
